(19) United States
(12) Patent Application Publication
(10) Pub. No.: US 2023/0133330 A1
(43) Pub. Date: May 4, 2023

(54) SYSTEMS AND METHODS FOR MEDICAL RESOURCE INTELLIGENCE

(71) Applicant: Avail Medsystems, Inc., Santa Clara, CA (US)

(72) Inventors: Daniel HAWKINS, Palo Alto, CA (US); Ravi KALLURI, San Jose, CA (US); Arun KRISHNA, Pleasanton, CA (US); Shivakumar MAHADEVAPPA, Fremont, CA (US)

(21) Appl. No.: 18/046,710

(22) Filed: Oct. 14, 2022

Related U.S. Application Data
(63) Continuation of application No. PCT/US21/28119, filed on Apr. 20, 2021.
(60) Provisional application No. 63/012,402, filed on Apr. 20, 2020.

(51) Int. Cl. G16H 40/20

(52) U.S. Cl. CPC G16H 40/20

(57) ABSTRACT

Systems and methods for medical resource intelligence are provided. Systems and methods are provided for identifying appropriate remote users to contact, and for assessing productivity of such remote users. Systems and methods are provided for forecasting usage of medical resources. Furthermore, performance may be tracked in accordance with one or more metrics with aid of a video collaboration system.

### CROSS-REFERENCE

This application is a continuation of International Patent Application PCT/US21/28119, filed on Apr. 20, 2021, which claims priority to U.S. Provisional Application No. 63/012,402, filed on Apr. 20, 2020, each of which is incorporated herein by reference in its entirety for all purposes.

### BACKGROUND OF THE INVENTION

Medical products may be used during medical procedures, and may be associated with vendors that provide the medical products. Traditionally, vendor representatives may be present in-person during the medical procedures where associated products are being used, to provide support. Such in-person support can be time consuming and costly, as it requires that the vendor representative physically make his or her way to the various locations of the related medical procedures.

Additionally, information about usage of medical products that are supported by vendor representatives are not effectively tracked. Oftentimes, medical products may be acquired by health care facilities without understanding the impact and necessity of the usage of the medical products.

### SUMMARY OF THE INVENTION

A need exists for improved systems and methods for medical resource intelligence. A need exists for systems and methods that allow for identifying appropriate remote users to contact, and assess productivity of such remote users. A further need exists for forecasting usage of medical resources. Additionally, a need exists for systems and methods that may track performance and one or more metrics by usage of a video collaboration system.

Aspects of the invention are directed to a method of forecasting usage of one or more medical resources, said method comprising: (a) collecting, with aid of one or more video or audio systems, images or audio transcripts of one or more medical resources at a health care location; (b) recognizing, with aid of one or more processors, the one or more medical resources based on the images or audio transcripts collected by the video or audio systems; (c) assessing, with aid of one or more processors, usage of the or more medical resources based on the images or audio transcripts collected by the video or audio systems; and (d) forecasting, with aid of one or more processors, usage of the or more medical resources based on usage data of the one or more medical resources from multiple health care locations.

In one aspect, the present disclosure provides a method of forecasting usage of one or more medical resources, said method comprising: collecting, with aid of one or more video or audio systems, images or audio transcripts of one or more medical resources at a health care location; recognizing, with aid of one or more processors, the one or more medical resources based on the images or audio transcripts collected by the video or audio systems; assessing, with aid of one or more processors, usage of the one or more medical resources based on the images or audio transcripts collected by the video or audio systems; and forecasting, with aid of one or more processors, usage of the one or more medical resources based on usage data of the one or more medical resources from multiple health care locations.

In some embodiments, forecasting the usage of the one or more medical resources comprises incorporating one or more health care facility or department rules for the health care location. In some embodiments, forecasting the usage of the one or more medical resources comprises incorporating usage data collected with aid of one or more medical consoles capable of communicating with a remote device. In some embodiments, forecasting usage of the one or more medical resources comprises real-time forecasting during a medical procedure at the health care location. In some embodiments, forecasting the usage of the one or more medical resources comprises incorporating scheduling data from a scheduling system. In some embodiments, forecasting the usage of the one or more medical resources comprises forecasting a higher usage of a medical product that has a lower cost than a practitioner's preference while providing equivalent or improved outcomes. In some embodiments, forecasting the usage of the one or more medical resources comprises predicting a future usage of a particular category of product. In some embodiments, forecasting the usage of the one or more medical resources comprises predicting a future usage of a specific model of product.

In some embodiments, the method may further comprise displaying a forecasted usage of the one or more medical resources to a member of a health care facility. In some embodiments, the method may further comprise displaying a forecasted usage of the one or more medical resources to a member of a vendor that provides the one or more medical resources.

In another aspect, the present disclosure provides a method of assessing productivity of a remote user providing support during a medical procedure, said method comprising: collecting, with aid of one or more video or audio systems, information pertaining to support provided by the remote user; and assessing, with aid of one or more processors, productivity of the remote user based on (i) the information provided by the one or more video and audio systems and (ii) prior support provided by the remote user. In some embodiments, the one or more video or audio systems comprise a medical console capable of communicating with a remote device of the remote user. In some embodiments, assessing the productivity of the remote user comprises analyzing data corresponding to an amount of time the remote user spends supporting the medical procedure. In some embodiments, the amount of time the remote user spends supporting the medical procedure is tracked by detecting a time at which communications are initiated between a medical console and a remote device of the remote user, and a time at which communications are completed between the medical console and the remote device of the remote user. In some embodiments, the amount of time the remote user spends supporting the medical procedure is tracked by detecting an amount of time during which the remote user is actively interacting with a remote device of the remote user.

In some embodiments, assessing the productivity of the remote user depends on a product or product type supported by the remote user. In some embodiments, assessing the productivity of the remote user depends on an outcome of the medical procedure utilizing the product or product type supported by the remote user. In some embodiments, assessing the productivity of the remote user depends on product usage over time for a product supported by the remote user. In some embodiments, the method may further comprise displaying a quantitative assessment of the remote user's productivity.

In another aspect, the present disclosure provides a system for assessing productivity of a remote user providing support during a medical procedure, said system comprising: one or more video or audio systems configured to collect information pertaining to support provided by the remote user; and one or more processors configured to assess, with aid of a machine learning system, productivity of the remote user based on (i) the information provided by the one or more video and audio systems and (ii) prior support provided by the remote user.

Additional aspects and advantages of the present disclosure will become readily apparent to those skilled in this art from the following detailed description, wherein only exemplary embodiments of the present disclosure are shown and described, simply by way of illustration of the best mode contemplated for carrying out the present disclosure. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an example of a video capture system, in accordance with embodiments of the invention.

FIG. 2A shows an example of a medical resource intelligence system configured to communicate with various devices, in accordance with embodiments of the invention.

FIGS. 2B-2E show examples of various machine learning techniques that may be utilized, in accordance with embodiments of the invention.

FIG. 3 shows an example of one or more factors that may be considered in identifying a vendor representative to provide support in a particular instance, in accordance with embodiments of the invention.

FIG. 4 shows an example of one or more factors that may be assessed in determining vendor representative productivity, in accordance with embodiments of the invention.

FIG. 5 shows an example of medical products that may be recognized using a video capture system, in accordance with embodiments of the invention.

FIG. 6 shows an example of a system for calculating usage of a medical resource, in accordance with embodiments of the invention.

FIG. 7 shows an example of a user interface displaying medical resource intelligence to a health care facility, in accordance with embodiments of the invention.

FIG. 8 shows an example of a user interface displaying medical resource intelligence to a vendor representative, in accordance with embodiments of the invention.

FIG. 9 shows an example of a user interface displaying medical resource intelligence to a manufacturer, in accordance with embodiments of the invention.

FIG. 10 shows an exemplary computer system, in accordance with embodiments of the invention.

### DETAILED DESCRIPTION OF THE INVENTION

While preferable embodiments of the invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

The invention provides systems and methods for medical resource intelligence. Various aspects of the invention described herein may be applied to any of the particular applications set forth below. The invention may be applied as a part of a health care system or communication system. It shall be understood that different aspects of the invention can be appreciated individually, collectively or in combination with each other.

The systems and methods provided herein may be useful for assessing appropriate remote users to contact for a medical procedure. One or more factors based on collected data may be useful for identifying a remote user to contact. Performance of one more individuals involved in a medical procedure may be assessed. For example, performance of a remote user, such as a vendor representative, may be assessed. This may advantageously improve a selection of remote users to support a procedure in the future. The systems and methods provided herein may also provide an assessment of outcomes and account for multiple factors that may provide an optimal or improved outcome.

In some embodiments, data collected during a medical procedure may be useful for forecasting product usage. The systems and methods provided herein may determine actual usage of products. The systems and methods provided herein may also be useful for determining usage of product based on the data and/or information about scheduling or arrangements with vendors for particular products. This may advantageously permit a health care facility to purchase an appropriate number of products, without over-purchasing or under-purchasing.

The systems and methods provided herein may utilize a video capture system in order to capture images during the surgical procedure. In some cases, the systems and methods provided herein may utilize a plurality of video capture systems in order to capture images or videos during the surgical procedure.

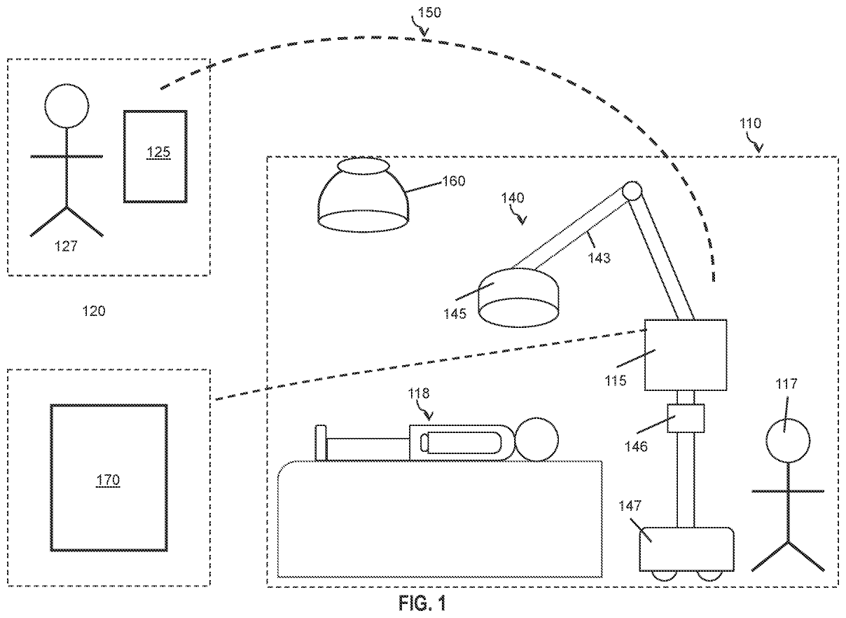

FIG. 1 shows an example of a video capture system utilized within a medical suite, such as an operating room. The video capture system may optionally allow for communications between the medical suite and one or more remote individuals, in accordance with embodiments of the invention. Communication may optionally be provided between a first location 110 and a second location 120.

The first location 110 may be a medical suite, such as an operating room of a health care facility. A medical suite may be within a clinic room or any other portion of a health care facility. A health care facility may be any type of facility or organization that may provide some level of health care or assistance. In some examples, health care facilities may include hospitals, clinics, urgent care facilities, out-patient facilities, ambulatory surgical centers, nursing homes, hospice care, home care, rehabilitation centers, laboratory, imaging center, veterinary clinics, or any other types of facility that may provide care or assistance. A health care facility may or may not be provided primarily for short term care, or for long-term care. A health care facility may be open at all days and times, or may have limited hours during which it is open. A health care facility may or may not include specialized equipment to help deliver care. Care may be provided to individuals with chronic or acute conditions. A health care facility may employ the use of one or more health care providers (a.k.a. medical personnel/medical practitioner). Any description herein of a health care facility may refer to a hospital or any other type of health care facility, and vice versa.

The first location may be any room or region within a health care facility. For example, the first location may be an operating room, surgical suite, clinic room, triage center, emergency room, or any other location. The first location may be within a region of a room or an entirety of a room. The first location may be any location where an operation may occur, where surgery may take place, where a medical procedure may occur, and/or where a medical product is used. In one example, the first location may be an operating room with a patient 118 that is being operated on, and one or more medical personnel 117, such as a surgeon or surgical assistant that is performing the operation, or aiding in performing the operation. Medical personnel may include any individuals who are performing the medical procedure or aiding in performing the medical procedure. Medical personnel may include individuals who provide support for the medical procedure. For example, the medical personnel may include a surgeon performing a surgery, a nurse, an anesthesiologist, and so forth. Examples of medical personnel may include physicians (e.g., surgeons, anesthesiologists, radiologists, internists, residents, oncologists, hematologists, cardiologists, etc.), nurses (e.g., CNRA, operating room nurse, circulating nurse), physicians' assistants, surgical techs, and so forth. Medical personnel may include individuals who are present for the medical procedure and authorized to be present.

Medical resources may include medical products, medical personnel, locations, instruments, utilities, or any other resource that may be involved for a medical procedure.

Medical products may include devices that are used alone or in combination with other devices for therapeutic or diagnostic purposes. Medical products may be medical devices. Medical products may include any products that are used during an operation to perform the operation or facilitate the performance of the operation. Medical products may include tools, instruments, implants, prostheses, disposables, or any other apparatus, appliance, software, or materials that may be intended by the manufacturer to be used for human beings. Medical products may be used for diagnosis, monitoring, treatment, alleviation, or compensation for an injury or handicap. Medical products may be used for diagnosis, prevention, monitoring, treatment, or alleviation of disease. In some instances, medical products may be used for investigation, replacement, or modification of anatomy or of a physiological process. Some examples of medical products may range from surgical instruments (e.g., handheld or robotic), catheters, endoscopes, stents, pacemakers, artificial joints, spine stabilizers, disposable gloves, gauze, IV fluids, drugs, and so forth.

Medical personnel may be considered as medical resources as well. For example, the number and types of individuals that may be required to be present at a medical procedure may be considered as a medical resource.

A video capture system may have one or more cameras. The video capture system may comprise one or more medical consoles. The video capture system may also comprise a local communication device 115. The local communication device may optionally communicate with a remote communication device 125. The local communication device may be a part of a medical console. The local communication device may be an integral part of the medical console or may be separable or detachable from the medical console. The remote communication device may be at a location separate from the medical console. In one example, a local communication device may be a smartphone or tablet that is supported or integrated into the medical console. In another example, the remote communication device may be a smartphone or tablet used by a remote user.

One or more cameras may be integral to a communication device, such as the local communication device or the remote communication device. Alternatively, the one or more cameras may be removable and/or connectable to the communication device. The one or more cameras may face a user when the user looks at a display of the communication device. The one or more cameras may face away from a user when the user looks at a display of the communication device. In some instances, multiple cameras may be provided which may face in different directions. The cameras may be capable of capturing images at a desired resolution. For instance, the cameras may be capable of capturing images at least a 6 mega pixel, 8 mega pixel, 10 mega pixel, 12 mega pixel, 20 mega pixel, 30 megapixels, 40 megapixels, or any number of pixels. The cameras may be capable of capturing SD, HD, Full HD, WUXGA, 2K, UHD, 4K, 8K, or any other level of resolution. A camera on a rep communication device may capture an image of a vendor representative. A camera on a local communication device may capture an image of a medical personnel. A camera on a local communication device may capture an image of a surgical site and/or medical tools, instruments or products.

The communication device may comprise one or more microphones or speakers. A microphone may capture audible sounds such as the voice of a user. A microphone array in the console may capture audio/speech in the room. For instance, the rep communication device microphone may capture the speech of the vendor representative and a local communication device microphone may capture the speech of a medical personnel. One or more speakers may be provided to play sound. For instance, a speaker on a rep communication device may allow a vendor representative to hear sounds captured by a local communication device, and vice versa.

In some embodiments, an audio enhancement module may be provided. The audio enhancement module may be supported by a video capture system. The audio enhancement module may comprise an array of microphones that may be configured to clearly capture voices within a noisy room while minimizing or reducing background noise. The audio enhancement module may be separable or may be integral to the video capture system.

A medical console may comprise a communication device. The communication device may be an integral part of the medical console or may be a separate part of the medical console. A communication device may comprise a display screen. The display screen may be a touchscreen. The display screen may accept inputs by a user's touch, such as an input provided by the user's finger. The display screen may accept inputs by a stylus or other tool.

A communication device may be any type of device capable of communication. For instance, a communication device may be a smartphone, tablet, laptop, desktop, server, personal digital assistant, wearable (e.g., smartwatch, glasses, etc.), or any other type of device.

In some embodiments, a local communication device 115 may be supported by a medical console 140. The local communication device may be permanently attached to the medical console, or may be removable from the medical console. Optionally, a local communication device may be an integral part of the medical console. In some instances, the local communication device may remain functional while removed from the medical console. The medical console may optionally provide power to the local communication device when the local communication device is attached to (e.g., docked with) the medical console. The medical console may be mobile console that may move from location to location. For instance, the medical console may include wheels that may allow the medical console to be wheeled from location to location. The wheels may be locked into place at desired locations. The medical device may optionally comprise a lower rack and/or support base 147. The lower rack and/or support base may house one or more components, such as communication components, power components, auxiliary inputs, and/or processors.

The medical console may optionally include one or more cameras 145, 146. The cameras may be capable of capturing images of the patient 118, or portion of the patient (e.g., surgical site). The cameras may be capable of capturing images of the medical devices. The cameras may be capable of capturing images of the medical devices as they rest on a tray, or when they are handled by a medical personnel and/or used at the surgical site. The cameras may be capable of capturing images at any resolution, such as those described elsewhere herein. The cameras may be used to capture a still images and/or video images. The cameras may be capturing images in real time.

One or more of the cameras may be movable relative to the medical console. For instance, one or more cameras may be supported by an arm. The arm may include one or more sections. In one example, a camera may be supported at or near an end of an arm. The arm may include one or more sections, two or more section, three or more sections, four or more sections, or more sections. The sections may move relative to one another or a body of the medical console. The sections may pivot about one or more hinge. In some embodiments, the movements may be limited to a single plane, such as a horizontal plane. Alternatively, the movements need not be limited to a single plane. The sections may move horizontally and/or vertically. A camera may have at least one, two, three, or more degrees of freedom. An arm may optionally include a handle that may allow a user to manually manipulate the arm to a desired position. The arm may remain in a position to which it has been manipulated. A user may or may not need to lock an arm to maintain its position. This may provide a steady support for a camera. The arm may be unlocked and/or re-manipulated to new positions as needed. In some embodiments, a remote user may be able to control the position of the arm and/or cameras.

In some embodiments, one or more cameras may be provided at the second location. The one or more cameras may or may not be supported by the medical console. In some embodiments, one or more cameras may be supported by a ceiling 160, wall, furniture, or other items at the second location. For instance, one or more cameras may be mounted on a wall, ceiling, or other device. Such cameras may be directly mounted to a surface, or may be mounted on a boom or arm. For instance, an arm may extend down from a ceiling while supporting a camera. In another example, an arm may be attached to a patient's bed or surface while supporting a camera. In some instances, a camera may be worn by medical personnel. For instance, a camera may be worn on a headband, wrist-band, torso, or any other portion of the medical personnel. A camera may be part of a medical device or may be supported by a medical device (e.g., endoscope, etc.). The one or more cameras may be fixed cameras or movable cameras. The one or more cameras may be capable of rotating about one or more, two or more, or three or more axes. The one or more cameras may include pan-tilt-zoom cameras. The cameras may be manually moved by an individual at the location. The cameras may be locked into position and/or unlocked to be moved. In some instances, the one or more cameras may be remotely controlled by one or more remote users. The cameras may zoom in and/or out. Any of the cameras may have any of the resolution values as provided herein. The cameras may optionally have a light source that may illuminate an area of interest. Alternatively, the cameras may rely on external light sources.

Images captured by the one or more cameras 145, 146 may be analyzed as described further elsewhere herein. The video may be analyzed in real-time. The videos may be sent to a remote communication device. This may allow a remote use to remotely view images captured by the field of view of the camera. For instance, the remote user may view the surgical site and/or any medical devices being used. The remote user may be able to view the medical personnel. The remote user may be able to view these in substantially real-time. For instance, this may be within 1 minutes or less, 30 seconds or less, 20 seconds or less, 15 seconds or less, 10 seconds or less, 5 seconds or less, 3 seconds or less, 2 seconds or less, or 1 second or less of an event actually occurring.

This may allow a remote user to lend aid or support without needing to be physically at the first location. The medical console and cameras may aid in providing the remote user with the necessary images and information to have a virtual presence at the first location.

The video analysis may occur locally at the first location 110. In some embodiments, the analysis may occur on-board a medical console 140. For instance, the analysis may occur with aid of one or more processors of a communication device 115 or other computer that may be located at the medical console. In some instances, the video analysis may occur remotely from the first location. In some instances, one or more servers 170 may be utilized to perform video analysis. The server may be able to access and/or receive information from multiple locations and may collect large datasets. The large datasets may be used in conjunction with machine learning in order to provide increasingly accurate video analysis. Any description herein of a server may also apply to any type of cloud computing infrastructure. The analysis may occur remotely and feedback may be communicated back to the console and/or location communication device in substantially real-time. Any description herein of real-time may include any action that may occur within a short span of time (e.g., within less than or equal to about 10 minutes, 5 minutes, 3 minutes, 2 minutes, 1 minute, 30 seconds, 20 seconds, 15 seconds, 10 seconds, 5 seconds, 3 seconds, 2 seconds, 1 second, 0.5 seconds, 0.1 seconds, 0.05 seconds, 0.01 seconds, or less).

In some embodiments, medical personnel may communicate with one or more remote individuals.

A second location 120 may be any location where a remote individual 127 is located. The second location may be remote to the first location. For instance, if the first location is a hospital, the second location may be outside the hospital. In some instances, the first and second locations may be within the same building but in different rooms, floors, or wings. The second location may be at an office of the remote individual. A second location may be at a residence of a remote individual.

A remote individual may have a remote communication device 125 which may communicate with a local communication device 115 at the first location. Any form of communication channel 150 may be formed between the rep communication device and the location communication device. The communication channel may be a direct communication channel or indirect communication channel. The communication channel may employ wired communications, wireless communications, or both. The communications may occur over a network, such as a local area network (LAN), wide area network (WAN) such as the Internet, or any form of telecommunications network (e.g., cellular service network). Communications employed may include, but are not limited to 3G, 4G, LTE communications, and/or Bluetooth, infrared, radio, or other communications. Communications may optionally be aided by routers, satellites, towers, and/or wires. The communications may or may not utilize existing communication networks at the first location and/or second location.

Communications between rep communication devices and local communication devices may be encrypted. Optionally, only authorized and authenticated rep communication devices and local communication devices may be able to communicate over a communication system.

In some embodiments, a remote communication device and/or local communication device may communicate with one another through a communication system. The communication system may facilitate the connection between the remote communication device and the local communication device. The communication system may aid in accessing scheduling information at a health care facility. The communication system may aid in presenting, on a remote communication device, a user interface to a remote individual about one or more possible medical procedures that may benefit from the remote individual's support.

A remote individual may be any user that may communicate remotely with individuals at the first location. The remote individual/user may lend support to individuals at the first location. For instance, the remote individual may support a medical procedure that is occurring at the first location. The remote user may provide support for one or more medical products, or provide advice to one or more medical personnel.

In some embodiments, the remote user may be a vendor representative. Medical products may be provided by one or more vendors. Typically, vendors may make arrangements with health care facilities to provide medical products. Vendors may be entities, such as companies, that manufacture and/or distribute medical products. The vendors may have representatives that may be able to provide support to personnel using the medical devices. The vendor representatives (who may also be known as product specialists or device reps), may be knowledgeable about one or more particular medical products. Vendor representatives may aid medical personnel (e.g., surgeons, surgical assistants, physicians, nurses) with any questions they may have about the medical products. Vendor representatives may aid in selection of sizing or different models of particular medical products. Vendor representatives may aid in function of medical products. Vendor representatives may help a medical personnel use product, or troubleshoot any issues that may arise. These questions may arise in real-time as the medical personnel are using a product. For instance, questions may arise about a medical product while a surgeon is in an operating room to perform a surgery. Traditionally, vendor representatives have been located at the first location with the medical personnel. However, this can be time consuming since the vendor representative will need to travel to the location of the medical procedure. Secondly, the vendor representative may be present but the vendor representative's help may not always be needed, or may be needed for a very limited time. Then, the vendor representative may have to travel to another location. It may be advantageous for a vendor representative to communicate remotely as needed with personnel at the first location. Thus, in systems and methods provided herein, the vendor representative may be a remote individual at a second location who may provide support remotely.

The remote users may be any other type of individual providing support, such as other medical personnel (e.g., specialists, general practice physicians, consultants, etc.), or technical support. Any description herein of vendor representatives may also apply to any other type of individual providing support, and vice versa.

In some embodiments, information about communications between remote users, such as vendor representatives, and the medical console (or any other device at the first location) may be collected and used for any of the processes described elsewhere herein. For instance, call data records may include one or more of the following: call start time, call end time, call duration, the identity of the individuals on the call (e.g., remote user identity, medical personnel identity such as identity of medical practitioner used to log into a medical console or the identities of all medical personnel present at a medical procedure), identity of the medical console making a call (e.g., each medical console may have a unique or semi-unique identity, which may or may not encode a health care facility identity and/or medical personnel identity), bandwidth on audio and video throughout the call, or any other factors. In some embodiments, factors, such as video or audio bandwidth may be indicative of the amount of activity that has occurred on the call. This may be indicative of the degree of active support provided by the vendor representative during the call.

A call quality may be provided for a call. For example, quantitative feedback, such as a numeral value for the call quality may be provided. For example, the remote user and/or on-site medical personnel may provide feedback about the quality of the call itself. In one example, a score between 1 and 5 may be provided for the call. The users (e.g., remote users and/or on-site medical personnel) may be given an opportunity to provide the score after the call has been completed. This may be useful for assessing the functionality of the systems and methods provided herein.

In some instances, feedback may be provided for vendor representative (or other remote user) performance by the medical personnel. For example, a score of 1 to 5 may be provided for the vendor representative performance. In some instances, a vendor representative may be able to provide a score for the vendor representative's experience working with particular medical personnel.

In the systems and methods provided herein, a medical resource intelligence system may gather information collected at one or more locations (e.g., first locations). The medial resource intelligence may utilize video information, audio information, information from instruments that may be connected to a medical console, or information input by one or more medical personnel.

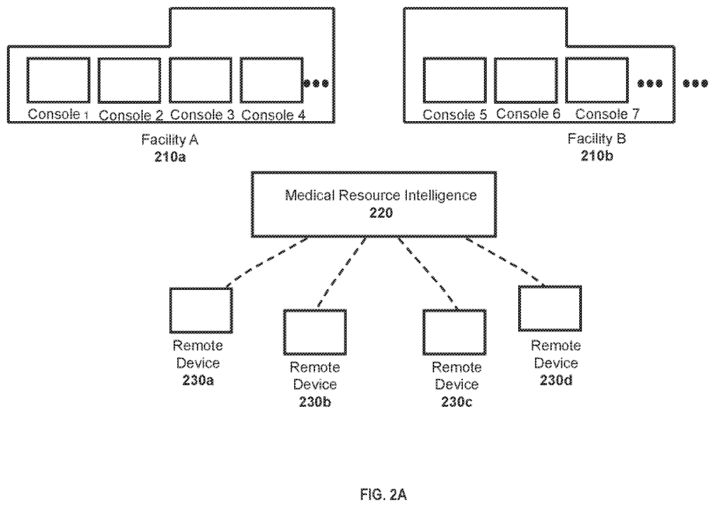

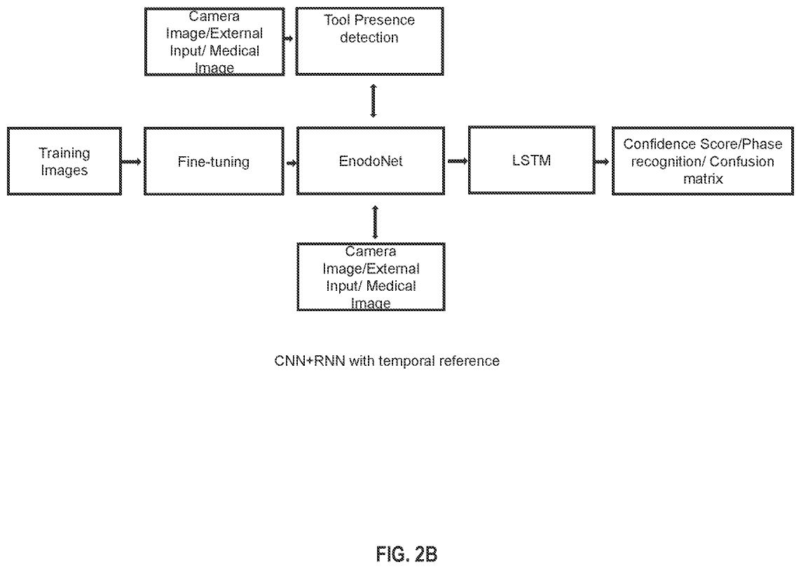

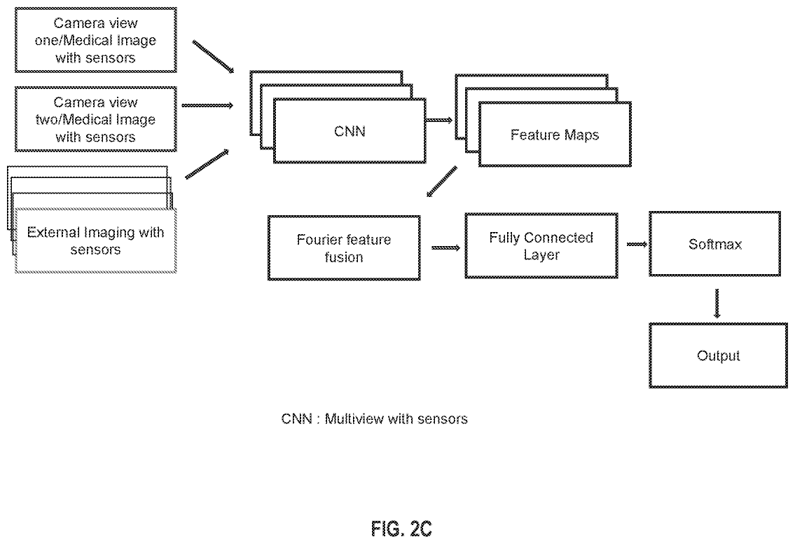

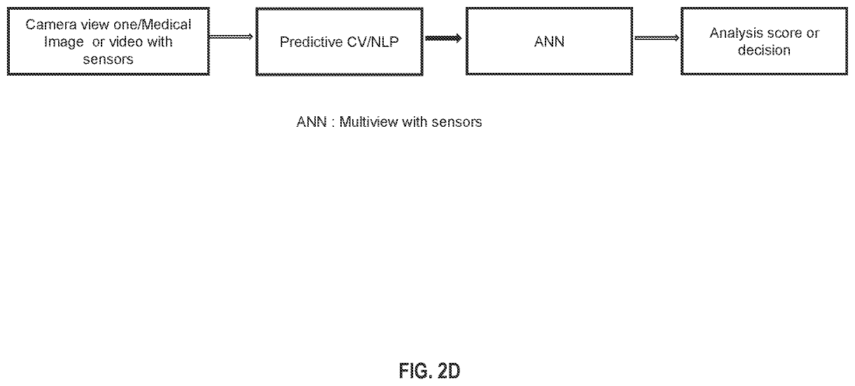

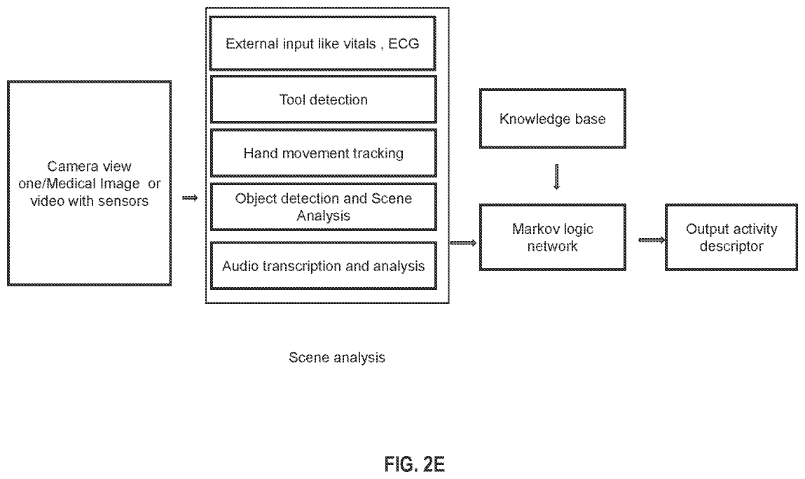

FIG. 2A shows an example of a medical resource intelligence system 220 configured to communicate with various devices, in accordance with embodiments of the invention.

In some embodiments, one or more health care facilities 210a, 210b may communicate with a medical resource intelligence system. The one or more health care facilities may be any type of health care facility such as those described elsewhere herein. The various types of health care facilities communicating with the medical resource intelligence system may be the same type of health care facility or different types of health care facilities. Such communications may occur simultaneously.

A health care facility may have one or more sources of information. Examples of sources of information may include one or more medical consoles. A medical console may be deployed at a location of a medical procedure. For example, one or more medical consoles may be deployed at an operating suite. In another example, one or more medical consoles may be deployed at a recovery room. One or more medical consoles may be deployed at an intensive care unit (ICU). One or more medical consoles may be deployed at a nurses' station. One or more medical consoles may be deployed in hallways. One or more medical consoles may be deployed at a clinician's office. In some instances, a plurality of consoles may be deployed anywhere throughout a health care facility. One or more of the consoles may be activated or in use throughout a health care facility. The one or more consoles may be actively communicating throughout a health care facility.

The one or more consoles may communicate with a medical resource intelligence system. The one or more consoles may communicate directly with a medical resource intelligence system. The one or more consoles may provide real-time updates to the medical resource intelligence system. In some embodiments, additional devices or information from the health care facilities may be provided to the medical resource intelligence system. For example, scheduling information, inventory information, usage information, terms of agreements with vendors, etc. may be provided to the medical resource intelligence system. Such information may be provided on a periodic basis (e.g., every week, every day, every several hours, every hour, every several minutes, every minute, every several seconds, every second, or every fraction of a second). Such information may optionally be provided to the medical resource intelligence system in real-time.

Optionally, the one or more medical consoles may not directly communicate with the medical resource intelligence system. The medical consoles may communicate with one or more system for the health care facility within which they operate. The health care facility systems may then provide relevant information to the medical resource intelligence system.

Any information provided to the medical resource intelligence system may be compliant with any regulations, such as regulations for privacy. Any communications to the medical resource intelligence system may be HIPAA compliant. In some embodiments, information may be automatically deleted or redacted as needed in order to comply with regulations or rules.

The medical intelligence resource system may also communicate with one or more remote devices 230a, 230b, 230c, 230d. The remote devices may be owned, operate, or used by one or more remote users. The one or more remote users may include vendor representatives, medical personnel, technical support, or any other type of remote user. The one or more remote devices may be any type of devices, such as tablets, smartphones, personal digital assistants, laptop computers, desktop computers, wearable devices, smart appliances, kiosks, or any other type of device.

In some embodiments, communications may occur between the consoles and the one or more remote devices. In some embodiments, the communications may occur directly between the consoles and the one or more remote devices. The communications may occur over a network, such as any type of network as described elsewhere herein. The existence, timing, duration, and identity of the devices (e.g., medical consoles or remote devices) may be logged and/or provided to the medical resource intelligence system. In some instances, the devices may include one or more geolocation device that may be used to determine the locations of the devices. The locations of the devices may be logged and/or provided to the medical resource intelligence systems as well.

The medical resource intelligence system may be implemented with aid of one or more devices. For instance, one or more servers may be utilized to implement the medical intelligence resource system. Optionally, one or more databases may be employed in order to store information. In some embodiments, cloud computing infrastructures may be utilized by the medical resource intelligence system. Optionally, peer-to-peer communications may be used in order to provide the functionality of the medical intelligence resource system. The medical resource intelligence system may utilize one or more processors, individually or collectively, that may perform any of the steps described herein. The medical resource intelligence system may utilize non-transitory, tangible computer readable media which may comprise memory storage units that may comprise code, logic, or instructions to perform any of the steps herein

The medical resource intelligence system may utilize machine learning to provide any of the functionality described herein. One or more data sets may be provided. Machine learning data may be generated based on the data sets. The learning data may be useful for medical resource intelligence, such as identifying and forecasting use of medical resources, identities of individuals to contact, evaluation of performance, performance metrics, video analysis, audio analysis, or other functions. The data from such functions may be fed back into the data sets to improve the machine learning algorithms.

One or more data sets may be provided. In some embodiments, data sets may advantageously include a large number of examples collected from multiple sources. In some embodiments, the medical resource intelligence system may be in communication with multiple health care facilities and may collect data over time regarding procedures. The data sets may include data about the patients, procedures performed, medical personnel involved, communications information (e.g., identity of remote users, locations, timing, duration, type of support provided, etc.) and associated timing information. As medical personnel perform additional procedures and communicate with remote users, data relating to these procedures and communications may be constantly updated and added to the data sets. This may improve the machine learning algorithm and subsequent predictions over time.

The one or more data sets may be used as training data sets for the machine learning algorithms. Learning data may be generated based on the data sets. In some embodiments, supervised learning algorithms may be used. Optionally, unsupervised learning techniques and/or semi-supervised learning techniques may be utilized in order to generate learning data.

In some embodiments, the machine learning may be used to improve object recognition. In some embodiments, video captured from one or more cameras during the medical procedure may be analyzed to detect medical products at the location. Optionally, audio data, medical records, or inputs by medical personnel may be used in addition or alternatively in order to determine medical products. In some embodiments, object recognition and/or sizing/scaling techniques may be used to determine the medical products at the location. A medical personnel may or may not provide feedback in real-time whether the medical product that was recognized using the video analysis was correct. In some embodiments, the feedback may be useful for improving medical product recognition in the future.

In some embodiments, the machine learning may be used to improve facial recognition or other ways of recognizing the identity and actions of the medical personnel. In some embodiments, video captured from one or more cameras during the medical procedure may be analyzed to detect identities of the medical personnel at the location. Similarly actions taken by the medical personnel may be recognized. Optionally, audio data, medical records, or inputs by medical personnel may be used in addition or alternatively in order to determine identity and/or actions of medical personnel. In some embodiments, object recognition and/or sizing/scaling techniques may be used to determine the medical personnel or their actions at the location. A medical personnel may or may not provide feedback in real-time whether the identities or actions that was recognized using the video analysis was correct. In some embodiments, the feedback may be useful for improving identity or action recognition in the future.

In some embodiments, the identity of the medical products that are actually used and timing of the use of the products during steps for a medical procedure may be predicted and/or recognized using a machine learning algorithm. In some embodiments, video information, audio data, medical records, and/or inputs by medical personnel may be used alone or in combination to determine the use of medical products and/or timing of use. Optionally, medical personnel may or may not provide feedback in real-time whether the identified use of the medical product and/or associated timing are correct. In some embodiments, the feedback may be useful for improving product use and timing predictions and/or recognition in the future.

As medical personnel are performing a medical procedure, the various steps for a medical procedure may be recognized using a machine learning algorithm. In some embodiments, video information, audio data, medical records, and/or inputs by medical personnel may be used alone or in combination to recognize the steps for the medical procedure that are being performed by the medical personnel. Machine learning may be useful for detecting and recognizing a series of steps for the procedure based on the collected information. Optionally, medical personnel may or may not provide feedback in real-time whether the detected steps are correct for the particular patient. In some embodiments, the feedback may be useful for improving step recognition in the future.

Similarly, during a medical procedure, the timing for the various steps for a medical procedure may be recognized using a machine learning algorithm. In some embodiments, video information, audio data, medical records, and/or inputs by medical personnel may be used alone or in combination to recognize the timing of the steps for the medical procedure that are being performed by the medical personnel. For instance, the systems and methods provided herein may recognize the time at which various steps are started. The systems and methods provided herein may recognize a length of time it takes for the steps to be completed. The systems and methods provided herein may recognize when the next steps are taken. Machine learning may be useful for detecting and recognizing timing for a series of steps for the procedure based on the collected information. Optionally, medical personnel may or may not provide feedback in real-time whether the timing of the detected steps are correct for the particular patient. In some embodiments, the feedback may be useful for improving step timing recognition in the future.

As described, machine learning may be useful for additional steps, such as recognizing individuals at the location (e.g., medical personnel) and items (e.g., medical products, medical devices) being used. The systems and methods provided may be able to analyze and identify individuals in the room based on the video frames and/or audio captured. For example, facial recognition, motion recognition, gait recognition, voice recognition may be used to recognize individuals in the room. The machine learning may also be utilized to recognize actions taken by the individuals (e.g., picking up an instrument, medical procedure steps, movement within the location). The machine learning may be utilized to recognize a location of the individual.

In some embodiments, the machine learning may utilize deep convolution neural networks/Faster R-CNN Nast NasNet (COCO). The machine learning may utilize any type of convolutional neural network (CNN) and/or recurrent neural network (RNN). Shift invariant or space invariant neural networks (SIANN) may also be utilized. Image classification, object detection and object localization may be utilized. Any machine learning technique known or later developed in the art may be used. For instance, different types of neural networks may be used, such as Artificial Neural Net(ANN), Convolution Neural Net (CNN), Recurrent Neural Net (RNN), and/or their variants.

The machine learning utilized may optionally be a combination of CNN and RNN with temporal reference, as illustrated in FIG. 2B. Input, such as cameras images, external inputs, and/or medical inputs may be provided to a tool presence detection module. The tool presence detection module may communicate with EnodoNet. Training images may be provided for fine-tuning, which may provide data to EnodoNet. Additional input, such as camera images, external inputs, and medical images may be provided to EnodoNet. The output from EnodoNet may be provided to long short-term memory (LSTM). This may provide an output of a confidence score, phase/step recognition, and/or confusion matrix.

The machine learning may optionally utilize CNN for Multiview with sensors as illustrated in FIG. 2C. In some embodiments, inputs, such as various camera views/medical images with sensors, and/or external imaging with sensors may be provided to a CNN learning module. This may provide output to feature maps, which may in turn undergo Fourier feature fusion. The data may then be conveyed to a fully connected layer, and then be provided to Softmax, and then be conveyed as an output.

In some embodiments, the machine learning as described and applied herein may be an artificial neural network (ANN) as illustrated in FIG. 2D. The Multiview with sensors may be provided as illustrated. For instance, an input, such as one or more camera views/medical image or video with sensors may be provided to a predictive (computer vision/natural language processing) CV/NLP module. The output may be conveyed to an ANN module. The output from the ANN may be an analysis score or decision.

FIG. 2E shows an example of scene analysis utilizing machine learning, in accordance with embodiments of the invention. An input may comprise one or more camera views and/or medical image or video with sensors. The input may be provided to a module that may perform one or more functions, such as external input like vitals (e.g., ECG), tool detection, hand movement tracking, object detection and scene analysis, and/or audio transcription and analysis. The output from the module may be provided to a Markov logic network. Data from a knowledge base may also be provided to a Markov logic network. The output from the Markov logic network may be an output activity descriptor.

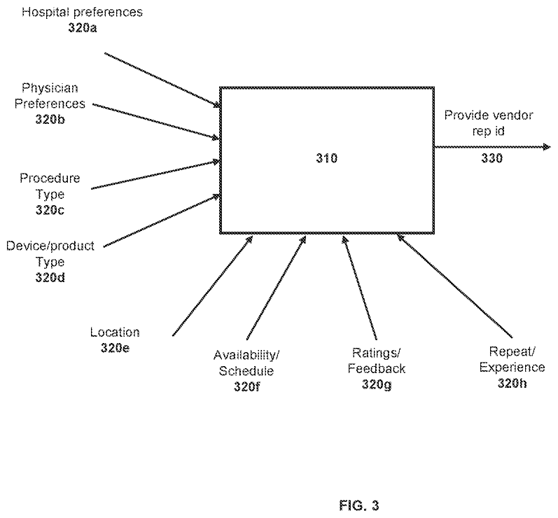

FIG. 3 shows an example of one or more factors that may be considered in identifying a vendor representative to provide support in a particular instance, in accordance with embodiments of the invention.

Various factors may be considered in identifying a vendor representative to provide support in a particular instance. Before or during a medical procedure, it may be desirable to identify a vendor representative to provide support. The medical console or other devices may be used to contact the vendor representative to provide support. The vendor representative support may be arranged before beginning the medical procedure. Alternatively, during the medical procedure, it may be determined that vendor representative support is needed, and an individual may be identified and contacted.

The various factors provided herein are provided by way of example only. Any other factors may be considered in the place of, or in addition to the factors provided herein. In some instances, not all of the factors illustrated herein are required. In some embodiments, 1, 2, 3, 4, 5, 6, 7, 8, 9, 10, 15, 20, 30, 50, or more of the factors provided herein are assessed in determining the identity of the vendor representative to provide support. Any number of factors, such as those more than any of the numbers provided, less than any of the numbers provided, or in between any of the numbers provided, may be considered.

A medical resource intelligence system 310 may assess any of the factors and provide a vendor representative identification 330 based on the factors.

An example of a factor that may be considered are health care facility preferences 320a. For examples, each hospital, clinic, care center, or any other type of health care facility may have preferred vendor representatives. In some embodiments, a health care facility may have a pool of approved vendor representatives. In some instances, health care facilities may have rankings or tiers (e.g., levels) of vendor representatives that are preferred. This may be based on arrangements with the vendor, past experience with the vendor representatives, recommendations, or any other factors.

Another example of a factor that may be considered are physician preferences 320a. For examples, each medical personnel may have preferred vendor representatives. For example, a surgeon may have vendor representatives he or she prefers from working with in the past. In some embodiments, a particular medical personnel may have a pool of approved or preferred vendor representatives. In some instances, medical personnel may have rankings or tiers (e.g., levels) of vendor representatives that are preferred. This may be based on arrangements with the vendor, past experience with the vendor representatives, recommendations, or any other factors.

Any description herein of healthcare facility preferences or physician preferences may also apply to department or group preferences. For example some departments or groups may have preferences for vendor representatives.

In some embodiments, physician preferences may depend on health care facility or group/department preferences. For example, if a health care facility only approves a particular pool of vendor representatives, the medical personnel may have preferences from within the pool of vendor representatives. In other examples, the health care facility may not be strict about the vendor representatives, but may have a preferred list, which the individual medical personnel may or may not choose to defer to.

In another example, procedure type 320c may be a factor that is considered. For example, particular vendor representatives may only support particular procedure types, or may have greater experience with certain procedure types. In some embodiments, the factor may be binary (e.g., vendor does or does not support a particular procedure type). Alternatively, the factor may have a greater gradient (e.g., may depend on the degree of experience the vendor representative has with particular procedure types, may depend on a preference ranking or score provided by the vendor representative to support particular procedure types).

Furthermore, medical product/device type 320d may be a factor to be considered. For example, particular vendor representatives may only support particular medical products, or may have greater experience with certain medical products. This may refer to categories of medical products (e.g., stents), or very particular medical products (e.g., stent model ABCD). In some embodiments, the factor may be binary (e.g., vendor does or does not support a particular medical product or product category). Alternatively, the factor may have a greater gradient (e.g., may depend on the degree of experience the vendor representative has with particular medical product or product category, may depend on a preference ranking or score provided by the vendor representative to support particular medical products or product categories).

Optionally, locations 320e may be considered as a factor. For examples, the locations of the various health care facilities may be known. The locations for the vendor representatives (real-time location, address on file, office location, residential location, etc.) may be known. In some embodiments, the relative locations between the health care facilities and the vendor representatives may be considered. In some embodiments, preferences may be given if they are closer together. In another example, preferences may be given for the same time zone or closer time zones. Alternatively, location may not be a factor, or may be a relatively weak factor when vendor representatives are providing support remotely.

In some embodiments, availability and scheduling 320f may be considered when identifying a particular vendor representative to provide support. In some embodiments, the vendor support may be needed at a particular point in time (e.g., scheduled operation, immediately upon request). In some embodiments, the vendor representative schedule may be known. In some instances, the vendor representative schedule may be pre-arranged. For example, the vendor may block out an hour if he or she knows that he or she will be busy supporting a particular procedure. The vendor representative schedule may optionally be updated in real-time. For example, if a vendor takes a remote call, the vendor representative schedule may be automatically updated to indicate that the vendor representative is not available, until the vendor representative is done with the call. In some instances, if the vendor representative is off working hours, or is unable to work, the vendor representative may provide an input that may indicate that the vendor representative is unavailable. The medical resource intelligence system may be synchronized with a vendor representative's calendar (e.g., Outlook calendar, Google calendar, or any other type of calendar) and may automatically be able to pull in some of the scheduling information. In some embodiments, the vendor representative availability may be known and updated in real-time. If the vendor representative is not available or is not predicted to available at the time needed, then the vendor representative may be removed from the pool of vendor representatives.

Similarly, if in the past, the vendor representative has shown him or herself to not be available when indicated as available, this may be a factor that may be considered. For example, if the vendor representatives has had numerous missed calls, the vendor representative may be less likely to be identified as the recommended vendor.

Optionally, ratings or feedback about the various vendor representatives 320g may be considered as a factor. For examples, personnel that have interacted with the vendor representatives before may provide feedback or other types of ratings or scores. For example, when a session is completed, a medical personnel may be presented with an opportunity to rate the experience working with the vendor representative. Quantitative (e.g., score, rating) and/or qualitative feedback (e.g., comments) may be collected and/or considered. The feedback may be aggregated and/or accumulated, which may be a factor in determining whether the vendor representative is identified. In some embodiments, the higher ratings/feedback will result in the vendor representative more likely being selected.

In some embodiments, the entirety of the feedback/ratings may be accumulated and used when identifying the vendor representative. In other instances, the feedback/ratings may be considered within the context, such as other factors. For example, a vendor representative may have a low rating for a particular medical product, but may have a high rating for a different medical product (e.g., second product). If the support requested is for the second product, the vendor representative may be assessed on the higher rating. In another example, if the vendor representative has a high rating from the individuals at a first health care facility and a low rating from individuals at a second health care facility, the ratings may be considered in the context of the health care facility requesting the support.

A vendor representative's experience 320h may be considered as a factor when making the determination. For instance, the vendor representative's experience at a particular health care facility may be considered (e.g., number of times the vendor representative has provided support, the duration of the support provided, etc.). In some instances, the experience with particular individuals or groups/departments at a facility may be considered (e.g., a vendor representative may have worked extensively with a particular surgeon, etc.). The vendor's experience supporting a particular product (e.g., product category, product model) may be considered. Similarly, the vendor's experience supporting a particular procedure or procedure type may be considered. The experience may be considered in conjunction with ratings. In some instances it may be desirable to use a vendor who has a large degree of experience with the product, procedure, personnel, and/or location.

In some embodiments, the outcomes may be considered in conjunction with the vendor experience. For example, when a vendor has supported a particular procedure type, the procedure types may be assessed to determine whether outcomes differ (e.g. are more positive, more negative) based on vendor support. If a vendor supporting a particular procedure type tends to result in a more favorable outcome for that procedure type, it may be more desirable to use that vendor again. In another example, when a vendor has supported a particular product, the procedures using that product may be assessed to determine whether outcomes differ (e.g. are more positive, more negative) based on vendor support. If a vendor supporting a particular product tends to result in a less favorable outcome for that procedures using that product, it may be less desirable to use that vendor again. In some embodiments, outcome may depend on the success of the procedure and/or how the patient recovers after the procedure. In some instances, outcome may depend on the length and/or efficiency of the procedure as well. For example, if a procedure takes much longer to complete than anticipated, this may be a less favorable outcome than a procedure that ended as scheduled.

The medical resource intelligence system 310 may consider any of the factors provided above. In some embodiments, the factors may be weighted equally in identifying one or more vendor representatives to support a particular procedure. Alternatively, different weights may be provided. For example, feedback/ratings may be rated higher than location. In some instances, one or more factors may be binary/prohibitive. For example, if a vendor representative is not available, the other factors may not matter, and then vendor representative may not be selected. In some instances, some factors may be given deference relative to other factors. For example, health care facility rules may trump individual physician preferences. The factors may be updated in real-time. The medical resource intelligence system may utilize machine learning to identify a vendor representative to support a procedure.

In some instances, the medical resource intelligence system may make a recommendation for a vendor representative based on the factors provided. The recommended vendor representative may be displayed to medical personnel or other individuals who may make the determination/confirmation whether to proceed with the recommended vendor representative. If the individual confirms the recommended vendor representative, then the vendor representative may be contacted and/or the schedule for the vendor representative may be updated for the procedure.

If the individual does not confirm the recommended vendor representative, the individual may be given the opportunity to select another vendor representative. The individual may directly enter the identity of the vendor representative the individual wishes to contact. In another example, a second recommended vendor representative option may be displayed to the individual, and so forth. The medical resource intelligence system may automatically rank recommended vendor representatives, and start at the top of the list, and move down as needed. For example, if contact is requested with the first choice vendor representative, but the contact is not made, the system may move on to the second choice vendor representative.

In some instances, the medical resource intelligence system may automatically attempt to connect with the identified vendor representative. For example, once the first choice vendor representative has been identified, the system may automatically attempt to connect with the vendor representative, or update the schedule of the vendor representative for a current or future procedure. If the connection with the vendor representative is unsuccessful, the system may automatically move on to the next choice vendor representative and so forth. This may occur without requiring user intervention or input.

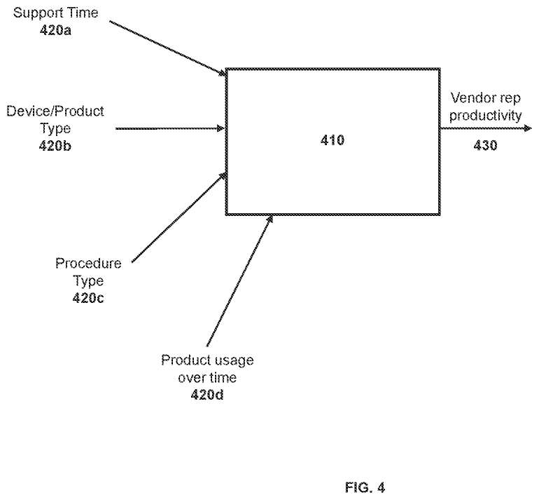

FIG. 4 shows an example of one or more factors that may be assessed in determining vendor representative productivity, in accordance with embodiments of the invention.

Various factors may be considered in determining vendor representative productivity. Vendor representative productivity may be updated in real-time as the vendor representative provides support. The vendor representative productivity may be updated periodically (e.g., every month, every week, every day, every several hours, every hour, every several minutes, every minute, every several seconds, every second, every fraction of a second, or any other degree of frequency).

The various factors provided herein are provided by way of example only. Any other factors may be considered in the place of, or in addition to the factors provided herein. In some instances, not all of the factors illustrated herein are required. In some embodiments, 1, 2, 3, 4, 5, 6, 7, 8, 9, 10, 15, 20, 30, 50, or more of the factors provided herein are assessed in determining the identity of the vendor representative to provide support. Any number of factors, such as those more than any of the numbers provided, less than any of the numbers provided, or in between any of the numbers provided, may be considered.

A medical resource intelligence system 410 may assess any of the factors and provide a vendor representative productivity measure 430 based on the factors.

An example of a factor that may be considered is support time 420a. This may include the amount of time the vendor actively spends supporting a procedure. In some embodiments, the time that communications are initiated and/or the time that the communications are completed may be tracked. The duration of each session may be recorded. In some embodiments, if a communication session is active, this may be considered the amount of time the vendor is supporting the procedure. If a vendor is simultaneously viewing multiple procedures, the vendor may be considered to be actively supporting each of the procedures. In other instances, the vendor may be considered to be actively supporting a procedure when the vendor is actively interacting with the communication (e.g., speaking, enlarging view, clicking on objects for that procedure, etc.).

Another factor that may be considered is the device or product type 420b that the vendor representative is supporting. For instance, the amount of time that the vendor representative is spending supporting a procedure may be divided or assessed based on the product that the vendor is supporting. In some instances, a vendor may be supporting multiple products that may be used in the procedure. The analysis may account for product type (e.g., stents) and/or more specific product groupings or models (e.g., Stent Model ABCD). Outcomes to the procedures that used the particular product may be assessed.

Similarly, procedure type 420c that the vendor representative is supporting may be considered as a factor. For instance, the amount of time that the vendor representative is spending supporting any procedure may be divided or assessed based on the type of procedure that the vendor is supporting. In some instances, the procedure type may be grouped with any level of specificity (e.g., department (gastrointestinal vs cardiothoracic), procedure types (e.g., inserting a stent), or specific procedures (e.g., inserting a stent using a particular technique). Outcomes to the procedures may be assessed. For examples, clinical outcomes to the patients and/or speed/efficiency of the procedure may be assessed.

Product usage 420d may be a factor that is considered in assessing vendor representative productivity. For example, the product usage over time may be tracked. If the product usage is increasing over time, for institutions or individuals with whom the vendor representative has interacted, then this may reflect favorably on the vendor representative productivity. However, if product usage is decreasing for institutions or individuals with whom the vendor representative has interacted, then this may reflect less favorably on the vendor representative.

The medical resource intelligence system 410 may consider any of the factors provided above. In some embodiments, the factors may be weighted equally in assessing vendor representative productivity. Alternatively, different weights may be provided. In some instances, one or more factors may be binary/prohibitive. In some instances, some factors may be given deference relative to other factors. The factors may be updated in real-time. The medical resource intelligence system may utilize machine learning to assess vendor representative productivity.

The medical resource intelligence system may provide one or more outputs relating to vendor productivity 430. In some instances, the medical resource intelligence system may make provide a quantitative assessment of the vendor representative productivity. In some instances, numerical values such as the amount worked, the number of procedures that the vendor representative was able to support, the number of products the vendor representative was able to support, the feedback provided, the outcomes from the support, the resulting change (if any) in usage of the product may be provided.

A vendor representative may be able to view one or more metrics relating to his or her own productivity. The vendor representative may be able to view a report or any other type of breakdown of his or her own productivity. The vendor representative may be able to view productivity by procedure type or product. The vendor representative may be able to view information over time, such as hours worked over time, or associated product usage change over time.

In some embodiments, a vendor administrator (e.g., employer of vendor representative) may be able to view productivity. The vendor administrator may be able to view productivity for individual vendor representatives, or for multiple vendor representatives, which may be aggregated. The vendor may be able to view productivity measures by procedure type or product. The vendor may be able to view individual or aggregated information over time, such as hours worked over time, or associated product usage change over time.

In some instances, productivity may be tracked for when vendor representatives only use the remote communication system. Alternatively, productivity may be tracked when vendor representatives do not use the remote communication system (e.g., when the vendor attends a procedure in person, etc.). In some instances, comparisons may be made between productivity when the vendor does use a remote communication system vs. not using a remote communication system. For example, metrics, such as efficiency, growth in sales of product, amount of time providing support, may be assessed as a comparison.

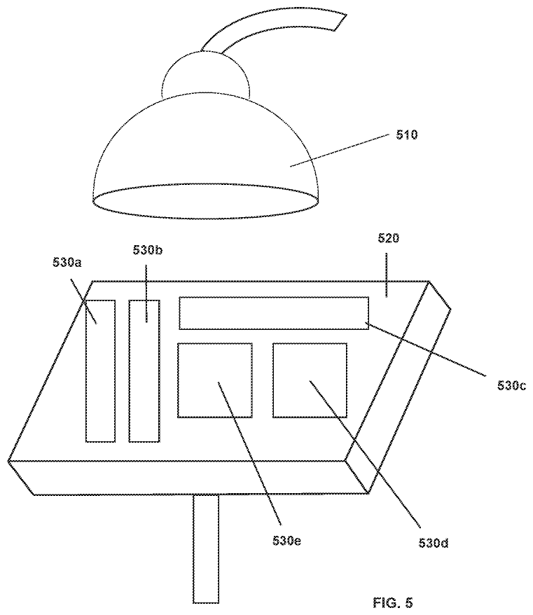

FIG. 5 shows an example of medical products that may be recognized using a video capture system, in accordance with embodiments of the invention.

As previously described, one or more cameras 510 may be provided at a location of a medical procedure. The one or more cameras may include cameras on a medical console, supported on a ceiling, a boom, an arm, a wall, furniture, worn by medical personnel, or any other location. Multiple cameras may optionally be provided. The video collected by the cameras may be aggregated and/or analyzed by a video analysis system.

The one or more cameras may individually or collectively capture images of the medical products 530a, 530b, 530c, 530d, 530e that may be used at the location. In one example, one or more cameras may individually or collectively capture an image of medical products that may be provided at a single location, such as a tray 520.

The video analysis system may be able to recognize the medical products that are provided. The medical product may be recognized in accordance with medical product type (e.g., stent), or may be recognized specifically to the model level (e.g., Stent Model ABCD manufactured by Company A). In some embodiments, the medical products may have graphical codes, such as QR codes, barcodes (e.g., 1D, 2D, 3D barcodes), symbols, letters, numbers, characters, shapes, sequences of lights or images, icons, or any other graphical code that may be useful for identifying the medical product. The cameras may capture images of the graphical codes, which may be useful for identifying the product type, specific product model, and/or specific product (e.g., tracked to the individual product, or batch/group).

In some embodiments, audio information may be collected as well. For example, speech by medical personnel may be analyzed to detect words that may refer to medical products. In some instances, the sound of medical products being used may be analyzed and recognized.

Medical records, surgeon prep cards, inputs by medical personnel, or any other sources may be used in recognizing the medical products that are provided at a procedure.

Additionally, the systems and methods provided herein (video, audio, records, prep cards, inputs, etc.) may be used to track usage of the medical products. For instance, the video may capture a medical personnel lifting a medical product and using it at a step during the procedure. The systems and methods provided herein may be able to recognize different steps of the procedure. The steps of the procedures may be predicted or known. In some instances, the steps of the procedure may provide context in trying to determine whether a particular medical product is being used. For example, if it is determined that a particular step is occurring, and that the step would require the use of a particular instrument, then the product that is imaged as being used may be interpreted within that context.

The timing and details regarding the actual use of the medical product may be recognized. Support given by a vendor representative at that time may also be recognized. In some embodiments, the timing and steps taken during the procedure may be used to determine efficacy of the product and/or support.

In some embodiments, the information may be collected passively without requiring any specialized input by medical personnel. For example, the images of the products may be automatically calculated and recognized.

Alternatively or in addition, medical personnel may provide some input or perform an action that may aid in detecting the products provided and/or used. In some instances, medical personnel may speak about the products that they are using. For example, as a medical personnel performs a step, the medical personnel may include information about the step and/or the product that is being used. One or more microphones (e.g., microphone array) may collect information and be able to translate the speech into text and/or recognize the products described.

In another example, medical personnel may scan the medical products to be used. For example, they may use a scanner to scan one or more graphical code provided on the product. This may occur prior to the medical procedure or at the beginning of the medical procedure. In some instances, scanning may occur as products are used as well to track the use of the products.

Optionally, the devices or wrappers for the devices may include RFID or other type of near field communication. One or more scanners or readers may be provided to detect the communications coming from the device to recognize product usage.

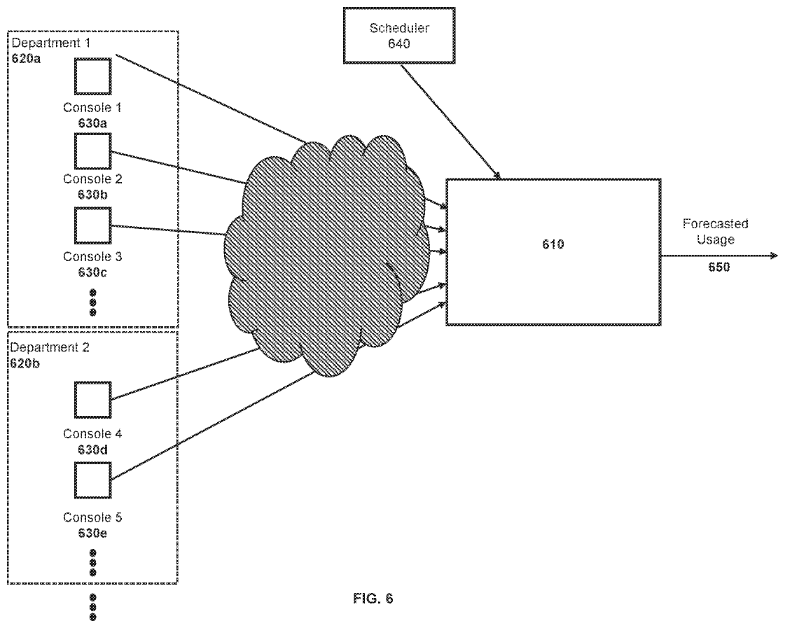

FIG. 6 shows an example of a system for calculating usage of a medical resource, in accordance with embodiments of the invention.

A medical resource intelligence system 610 may receive information that has been collected with aid of one or more source from a health care facility. The information may include information that has been collected with aid of one or more medical consoles 630. The medical console may have one or more features as described elsewhere herein. The medical console may include one or more cameras, microphones, communication devices, interfaces, auxiliary ports, servers, etc. The one or more sources may include one or more devices that are located at a site of a medical procedure. These may include other imaging devices, microphones, medical instruments or products, inputs, or other any other sources. Any description herein of medical consoles may apply to any other sources, and vice versa.

As previously described, information from medical consoles may be directly provided to the medical resource intelligence system. Alternatively or in addition, information from the medical consoles may be provided to a system of the health care facility, such may process the information before determining which information is provided to the medical resource intelligence system.

The information provided to the medical resource intelligence system may include information about medical products that may be used at the various locations. For example, the information may include the medical products that are present for a procedure. These may include products that have been prepared on a tray. These may include products that are indicated by a surgeon prep card.

Information about usage of the products during the procedure may be collected and provided to the medical resource intelligence system. For example, the systems and methods provided may be configured to recognize when a product is actually used during a procedure. The systems and methods provided may make note when a user interacts with a product. The systems and methods provided may make note when a user touches a product. The systems and methods provided herein may make note of the step of a medical procedure at which a user interacts with a product. The systems and methods provided may make note of the timing at which a user interacts with a product (e.g., time within the step, time within the procedure). The medical resource intelligence system may recognize when a product is not used during a procedure. For example, a product may be laid out for a procedure, but the medical personnel may end up leaving it on the tray or not interacting with it during the procedure. The medical resource intelligence system may also note when products are used that were not initially laid out or prepared for the procedure. For example, during the procedure, the medical personnel may request a particular item that was not initially prepared, and an individual may need to go and retrieve the product. The medical resource intelligence system may note when such a product is used, along with any associated details (e.g., the step of the procedure at which the product is used, the timing of the product usage).

In some embodiments, the information from the procedures about product usage may be provided from the entirety of the health care facility. In some instances, different groups, such as departments 620 within the health care facility may be able to individually collect or view product usage.

Information from the health care facility, such as a scheduler 640 may be provided to a medical resource intelligence system. For example, information about upcoming procedures may be provided to the medical resource intelligence system. The information about upcoming procedures may be useful for determining the products or types of products that may be used in such procedures. Such predictions may be made based on guidelines or facility/group/department preferences. For example, for Medical Procedure type ABCD, products type 1, 2, 3, and 4 may typically be required. In some instances, the predictions may be made based on specific medical personnel that may be performing the procedure. For example, different medical personnel may have different preferences for products. The scheduler may indicate the identity of the medical personnel slated to perform the procedure, and the forecasted usage of products may be made based on the preferences of the specific medical personnel.

Any description herein of information from a scheduler may include any other systems of a health care facility. This may include information about existing contracts or arrangements with vendors of medical products. The systems and methods provided herein may take information about the existing arrangements into account when forecasting usage. For example, if a certain equivalent product by a certain vendor will cost less, but provide similar or better clinical outcomes, the forecasting arrangement may forecast a higher usage of the less costly product. The forecasting arrangement may take into account any facility or department rules in connection with the vendor arrangements (e.g., will they enforce medical personnel using the less costly product, etc.).

Based on the information provided from one or more sources, such as consoles, and additional sources, such as a scheduler, the medical resource intelligence system may be able to forecast usage 650 of one or more medical products. For example, the medical resource intelligence may be able to predict the number of medical products will be used or needed in the future. In one example, the medical resource intelligence system may be able to predict a particular category of product, or specific model of product. The medical resource intelligence system may be able to predict, for example, how many unite of Stent ABCD manufactured by Vendor XYZ will be needed.

In some embodiments, the medical resource intelligence system may forecast the number of specific medical products to be used or needed in the future based on past information. In some embodiments, the predictions may be for particular products/models based on past usage and the assumption that the same product would be used for the same medical personnel, personnel type, department, or any other factor. In some embodiments, the medical resource intelligence system may be able to detect equivalents and the forecasted usage may be provided taking additional factors into account. For example, Stent Model ABCD by Vendor XYZ may be functionally equivalent to Stent Model 1234 by Vendor 789. The forecasted usage system may take into account vendor arrangements that may indicate how functionally equivalent models of products may be allocated in the future. For example, if a particular model from a vendor may be less costly in the future compared to the equivalent model, then the forecast may take this into account, and provide a higher forecast for the less costly model. The medical personnel preferences, group preferences, and/or health care facility preferences may be taken into account. For example, a health care facility may override individual medical personnel preferences in certain situations and only allow certain models to be available. In other instances medical personnel preferences may trump group or health care facility guidelines.

The forecasted usage of the medical products may extend out to any length of time. For example, a forecast may be provided for the next hour, the next several hours, the next day, the next several days, the next week, the next month, the next quarter, the next year, or any other length of time. The forecasted usage may be updated in real-time as information is collected. The system may receive information that may affect forecasted usage, from one or more sources, such as medical consoles in real-time as products are used, or at the end of every procedure, or any other degree of frequency. The system may receive information from schedulers, or any other health care facility system (e.g., with vendor arrangements) that may affect the forecasting of units.

The information relating to forecasted united may be provided to the health care facility. The health care facility may receive information on forecasted units needed for the entirety of the health care facility. In some instances, the information may be broken up into one or more groups, such as one or more departments. In some instances, each department may be able to see its own forecasted numbers. Any level or layer of groups may be provided for forecasting.

The forecasted usage may be provided with a high degree of accuracy. The substantially real-time updates, along with information from a scheduler or any other health care facility system may be useful for providing up-to-date forecasted usage that may be used to allow a health care facility to allocated resources.

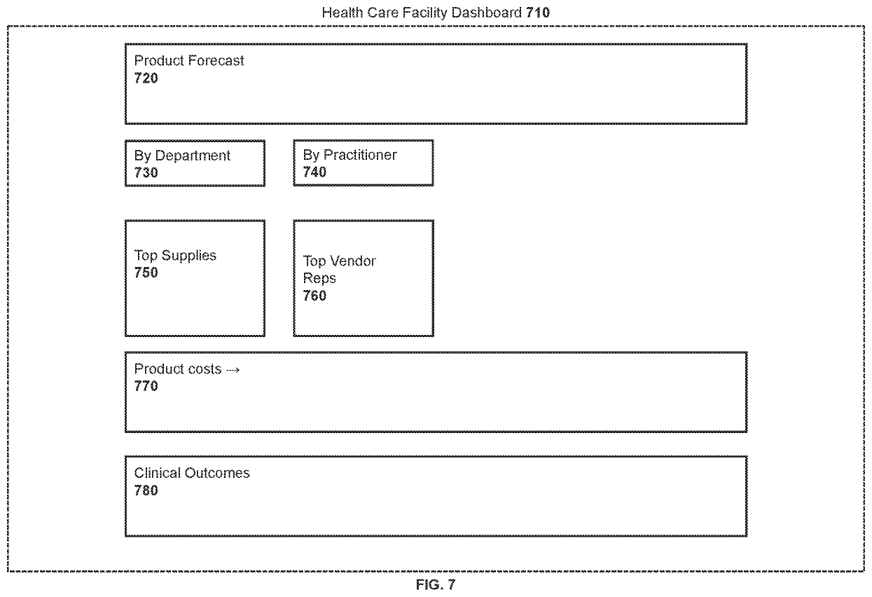

FIG. 7 shows an example of a user interface displaying medical resource intelligence to a health care facility 710, in accordance with embodiments of the invention.

Any type of medical resource intelligence that may be useful to the health care facility may be displayed on the user interface. The categories provided herein are provided by way of example only, and other types of medical resource intelligence data may be provided in any format. Furthermore, the arrangements provided herein are provided by way of example only, and alternative arrangements may be used. The placement and/or manner in displaying particular intelligence may be displayed. For example, they may be displayed as text, bar graphs, line graphs, pie charts, histograms, symbols, or any other arrangement.

A product forecast 720 may be provided for a health care facility. For example, for a particular medical product (e.g., product category, specific product model), a forecasted usage may be displayed. Past usage may or may not be displayed as well. The product usage over time (past, present, and/or future) may be displayed. A user may be able to select which product forecast is displayed. The usage may be able to select a product category or specific product model for which the forecast is displayed. The forecast may change as information is provided in real-time.

In some instances, the product forecast may be provided across the entirety of the health care facility. In some embodiments, the product forecast may be displayed by department 730. For example, a user may select a department to view the product forecast. For example, a product forecast may be provided for a gastrointestinal department, which may differ form a cardiothoracic unit. Similarly, the product forecast may include information about past usage. Usage between different groups, such as different departments at a healthcare facility may be displayed and/or compared.

In some embodiments, a user may toggle between views of product usage (and/or forecast) for different departments. In some instances, the views of product usage (and/or forecast) may overlay one another and/or be viewed simultaneously.

In some instances, a product forecast may be drilled down with greater specificity. For example, a product forecast may be displayed by practitioner 740. For example, a user may select a particular medical practitioner to view the product forecast. For example, a product forecast may be provided for Surgeon A, which may differ from a product forecast for Surgeon B. The product forecasts may differ based on upcoming surgeries (e.g., medical procedure types), and/or individual medical personnel preferences. Similarly, the product forecast may include information about past usage. Usage between different medical practitioners may be displayed and/or compared.

In some embodiments, a user may toggle between views of product usage (and/or forecast) for different medical practitioners. In some instances, the views of product usage (and/or forecast) may overlay one another and/or be viewed simultaneously.

In some instances, information about top suppliers 750 may be presented to the health care facility. For example, top vendors that provide products to the health care facility may be displayed to the health care facility. The top vendors may be calculated based on the number of units that are purchased and/or the total cost of purchases. The top vendors may be displayed so that they are ranked, with the vendor that provides the most units or has the largest amount of cost is displayed at the top. This may advantageously permit a health care facility to focus attention on vendors with which a lot of business is already conducted, which may be useful for purposes of re-negotiating contracts or making other decisions.

In some instances, information about top vendor representatives 760 may be presented to the health care facility. For example, top vendor representatives that provide support to the health care facility may be displayed to the health care facility. The top vendor representatives may be calculated based on the number of procedures that are supported, the number of products that have been covered, the length of time that the vendor representative has provided support, the clinical outcomes for procedures where the vendor representatives provide support, feedback or ratings for the vendor representatives, or any other factors or combinations thereof. In some instances, one or more of the factors may be weighted and used to determine a list of top vendor representatives. The top vendor representatives may be displayed so that they are ranked, with the vendor representatives that provide the most and/or best support is displayed at the top. This may advantageously permit a health care facility to focus attention on vendor representatives with whom a lot of positive interaction is already conducted, which may be useful for purposes of providing approved or preferred lists of vendor representatives, or making other decisions.

In some instances, information about outcomes 780 that may have utilized particular medical products or received particular vendor support may be displayed. The outcomes may include clinical outcomes, such as how the patient fared after the procedure, the amount of time that the procedure took, the cost for performing the procedure, or any other factors. This information may be compared to a mean value or norm. The deviation from the mean or norm may be determined. For example, for a particular medical product, if the patient heals more quickly after using a particular first medical product vs. another second medical product, the health care facility may prefer using the first medical product instead of the second medical product. The health care facility may make recommendations or rules to various departments or medical practitioners to use the first medical product instead of the second medical product.

Such information may be collected and displayed for various products. A user may be able to toggle between views of outcomes relating to different products. In some instances, the outcomes for multiple products may be provided as an overlay so that the outcomes for multiple products can be displayed simultaneously.

Such information may be collected and displayed for various vendor representatives. A user may be able to toggle between views of outcomes relating to different vendor representatives. In some instances, the outcomes for multiple vendor representatives may be provided as an overlay so that the outcomes for multiple vendor representatives can be displayed simultaneously.

The schematic of the user interface is provided by way of example only. Any of the data described herein may be displayed in each of their own visually discernible regions. The regions may be provided adjacent to one another, or above or below one another. The regions may be displayed simultaneously on the same user interface. The regions may have a fixed location or variable location. The regions may have any size or shape. The regions may have any placement relative to one another and are not limited to those provided herein.

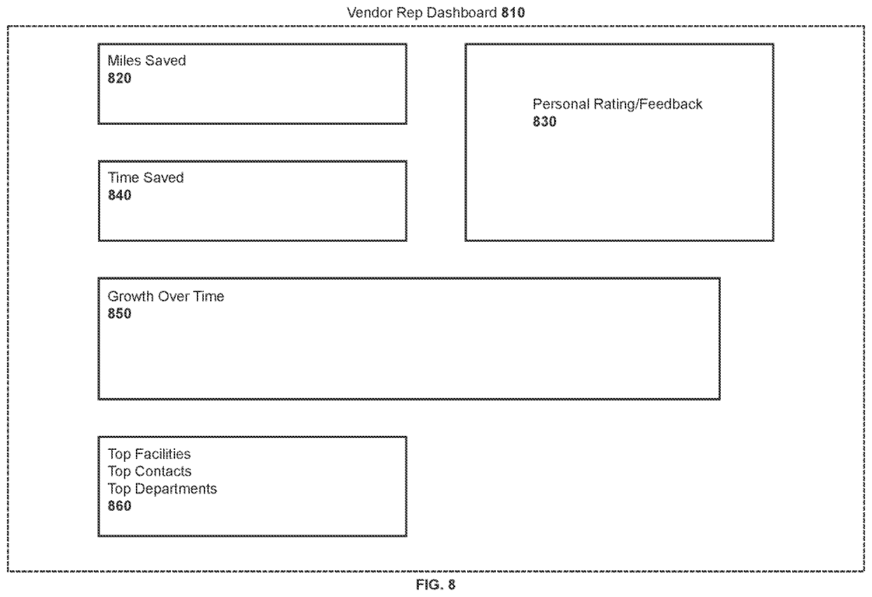

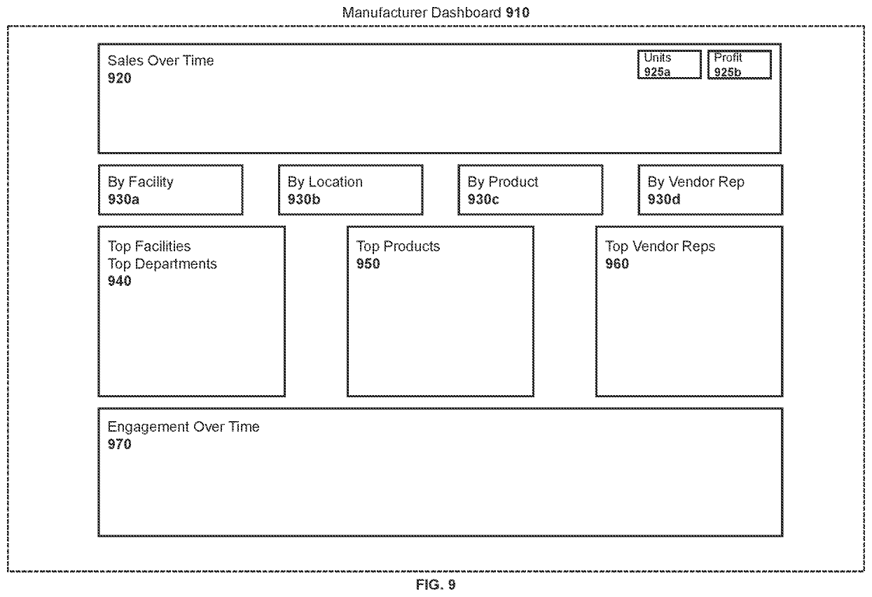

FIG. 8 shows an example of a user interface displaying medical resource intelligence to a vendor representative 810, in accordance with embodiments of the invention.

Any type of medical resource intelligence that may be useful to the vendor representative may be displayed on the user interface. The categories provided herein are provided by way of example only, and other types of medical resource intelligence data may be provided in any format. Furthermore, the arrangements provided herein are provided by way of example only, and alternative arrangements may be used. The placement and/or manner in displaying particular intelligence may be displayed. For example, they may be displayed as text, bar graphs, line graphs, pie charts, histograms, symbols, or any other arrangement.

Information about distance saved from travel by utilizing a remote system 820 may be provided for a vendor representative. For example, a vendor representative may provide support to one or more health care facility remotely. By not being required to travel to the health care facility, the vendor representative may save on distance traveled, which may also save time and allow the vendor representative to provide more support. The distance saved may be provided relative to any location (e.g., the vendor representative's home, the vendor representative's office, or any other reference location). The distance saved may be calculated in miles, kilometers, or any other unit of distance.

Information about time saved by utilizing a remote system 840 may be provided for a vendor representative. For example, a vendor representative may provide support to one or more health care facility remotely. By not being required to travel to the health care facility, and undergo possible physical processing and delay, the vendor representative may save time, which may allow the vendor representative to provide more support. The time saved may be calculated based on distance saved. In some instances, the time saved may depend on the amount of time that it would take to travel the distance. In some instances, map or traffic information may be utilized in order to determine the amount of time that was saved by not having to travel between particular locations at particular points in time. In some instances, information such as traffic data from the time of the procedure may be used to accurately calculate how much time was saved. Alternatively, more general information, such as speed limits, etc. may be utilized in calculating time saved by not traveling a particular distance. Other factors may be used to calculate time saved. For instance, the time to find parking and check in at a health care facility, other factors that may typically, add time required for a vendor representative to perform in-person support. The time saved may be calculated in weeks, days, hours, minutes, seconds, or any other unit of time.

A vendor representative may be able to view a personal rating and/or feedback 830. For example, after a vendor representative provides support for a procedure, associated medical personnel may be able to provide a rating and/or feedback. The rating may be a quantitative measure. For example, the vendor representative may receive a numerical score, such as a rating from 1 to 5, or between any other numbers. In another example, the vendor representatives may receive a letter grade, such as A through E. A vendor representative may receive an aggregate rating or score based on the ratings supplied for the various procedures that vendor representative has provided. The score may be based on the entire history of ratings provided to the vendor representative. In other instances, the sore may be based on the ratings provided within a particular timeframe (e.g., within the past several years, within the past year, within the past quarter, within the past month, etc.). In some instances, a vendor representative may be able to view a score for a particular product that the vendor representative has provided support, a particular medical procedure, a particular health care facility, or particular medical personnel that the vendor has provided support. Any of this information may be indexed, and the medical personnel may be able to view a score in relation to any of these parameters, or others.

In some instances, the vendor representative may receive qualitative feedback. For example, medical personnel may leave comments about the vendor representative's performance. This may include positive and/or negative comments. The vendor representative may be able to view the comments in order to understand areas where the vendor representative could improve, as well as areas of strength.

A vendor representative growth over time 850 may be displayed to the vendor representative. For example, for a particular vendor representative, past amount of support (e.g., length of time providing support, number of medical procedures supported, revenue generated, etc.) may be displayed. Forecasted amount of support may or may not be displayed as well. The vendor representative support over time may be displayed.

The vendor representative may be able to view growth/support amount over time with any level of specificity. For example, the vendor representative may be able to view growth for a particular product that the vendor representative supports. The vendor representative may potentially support multiple products. In some embodiments, a user may toggle between views of growth (and/or forecast) for different products. In some instances, the views of growth (and/or forecast) may overlay one another and/or be viewed simultaneously.

In another example, the vendor representative may be able to view growth for a particular procedure type that the vendor representative supports. The vendor representative may potentially support multiple procedure types. In some embodiments, a user may toggle between views of growth (and/or forecast) for different procedure types. In some instances, the views of growth (and/or forecast) may overlay one another and/or be viewed simultaneously.

In a further example, the vendor representative may be able to view growth for a particular health care facility, department, or individual medical personnel that the vendor representative supports. The vendor representative may potentially support multiple health care facilities, departments, or medical personnel. In some embodiments, a user may toggle between views of growth (and/or forecast) for different health care facilities, departments and/or medical personnel. In some instances, the views of growth (and/or forecast) may overlay one another and/or be viewed simultaneously.

In some instances, information about top facilities, top contacts ,and/or top departments 860 may be presented to vendor representative. For example, top facilities that use the vendor representative's services may be presented to the vendor representative. The top facilities may be calculated based on the number of products supported, the number of procedures supported, the length of time of support provided, or any other factor. The top facilities may be displayed so that they are ranked, with the facility that vendor representative provides the most support to is displayed at the top. This may advantageously permit a vendor representative to focus attention on facilities with which a lot of business is already conducted, which may be useful for purposes of allocating resources or making other decisions.

Similarly, top depai intents or top medical personnel that use the vendor representative's services may be presented to the vendor representative. The top departments and/or medical personnel may be calculated based on the number of products supported, the number of procedures supported, the length of time of support provided, or any other factor. The top departments or medical personnel may be displayed so that they are ranked, with the department or medical personnel that vendor representative provides the most support to is displayed at the top. This may advantageously permit a vendor representative to focus attention on departments or medical personnel with which a lot of business is already conducted, which may be useful for purposes of allocating resources or making other decisions.

The schematic of the user interface is provided by way of example only. Any of the data described herein may be displayed in each of their own visually discernible regions. The regions may be provided adjacent to one another, or above or below one another. The regions may be displayed simultaneously on the same user interface. The regions may have a fixed location or variable location. The regions may have any size or shape. The regions may have any placement relative to one another and are not limited to those provided herein.

FIG. 9 shows an example of a user interface displaying medical resource intelligence to a manufacturer 910, in accordance with embodiments of the invention.

Any type of medical resource intelligence that may be useful to the manufacturer or vendor may be displayed on the user interface. Any description herein of a manufacturer entity may apply to any type of vendor entity and vice versa. For example, the manufacturer may be any entity that makes and/or sells the medical products. The manufacturer may make and/or sell the medical products to intermediaries or to health care facilities.

The categories provided herein are provided by way of example only, and other types of medical resource intelligence data may be provided in any format. Furthermore, the arrangements provided herein are provided by way of example only, and alternative arrangements may be used. The placement and/or manner in displaying particular intelligence may be displayed. For example, they may be displayed as text, bar graphs, line graphs, pie charts, histograms, symbols, or any other arrangement.

Sales over time 920 may be provided for a manufacturer and/or vendor. For example, for a particular medical product (e.g., product category, specific product model), sales over time may be displayed. Predicted sales (e.g., usage) may or may not be displayed as well. The product sales over time (past, present, and/or future) may be displayed. A user may be able to select which product sales is displayed. The user may be able to select a product category or specific product model for which the forecast is displayed. The sales may change as information is provided in real-time.

The sales numbers may be displayed according to units 925a of sales. For example, the number of units of product sold or projected to be sold may be displayed. The sales numbers may be displayed according to profit or revenue 925b. For example, the profit/revenue from the units of product sold may be displayed.

In some instances, the sales information may be provided across the entirety of the manufacturer products at all locations. In some embodiments, the sales information may be displayed by health care facility 930a. For example, a user may select a health care facility to view the sales information for that particular health care facility. For example, a sales information may be provided Hospital ABC, which may differ from Hospital DEF. Usage between different facilities, such as different healthcare facilities may be displayed and/or compared.

In some embodiments, a user may toggle between views of sales history (and/or forecast) for different health care facilities. In some instances, the views of sales history (and/or forecast) may overlay one another and/or be viewed simultaneously.

In some instances, sales information may be based on location. For example, sales information may be displayed by location 930b. For example, a user may select a particular location to view the sales information. For example, sales information may be provided for Region A, which may differ from sales information for Region B. The location may have any level of specificity, such as by country, by region, by province, by state, by metropolitan area, by city, by neighborhood, or by any other level. Sales information between different locations may be displayed and/or compared.

In some embodiments, a user may toggle between views of sales information for different locations. In some instances, the views of sales information may overlay one another and/or be viewed simultaneously.

In some instances, sales information may be based on product. For example, sales information may be displayed by product 930c. A product may refer to a product category (e.g., stents) or specific product model (e.g., Stent Model ABCD). For example, a user may select a particular product to view the sales information. For example, sales information may be provided for Product A, which may differ from sales information for Product B. Sales information between different products may be displayed and/or compared.

In some embodiments, a user may toggle between views of sales information for different products. In some instances, the views of sales information may overlay one another and/or be viewed simultaneously.

In some instances, sales information may be based on vendor representative. For example, sales information may be displayed by vendor representative 930d. For example, a user may select a particular vendor representative to view the sales information. For example, sales information may be provided for Vendor Representative A, which may differ from sales information for Vendor Representative B. Viewing the information by vendor representative may allow for the manufacturer to see how the vendor representatives are performing. Sales information between different vendor representatives may be displayed and/or compared.

In some embodiments, a user may toggle between views of sales information for different vendor representatives. In some instances, the views of sales information may overlay one another and/or be viewed simultaneously.

Any of these factors or parameters may be combined. For example, any level of filtering may occur for any of the factors described elsewhere herein. For instance, a user may decide to view information by particular facility and by particular product.

In some instances, information about top facilities, top contacts, and/or top departments 940 may be presented to a manufacturer. For example, top facilities, contacts, and/or departments that use the manufacturer's services may be presented to the manufacturer. The top facilities, contacts, and/or departments may be calculated based on the number of products sold, the number of the profit or revenue from the sold products, or any other factor. The top facilities, contacts, and/or departments may be displayed so that they are ranked, with the facility, contact, and/or department that the manufacturer has the most sales with is displayed at the top. This may advantageously permit a manufacturer to focus attention on facilities, departments, or contacts with which a lot of business is already conducted, which may be useful for purposes of allocating resources or making other decisions.

Similarly, information about top products 950 may be presented to a manufacturer. For example, top products that are sold may be presented to the manufacturer. The top products may be calculated based on the number of products sold, the number of the profit or revenue from the sold products, or any other factor. The top products may be displayed so that they are ranked, with the products that the manufacturer has the most sales is displayed at the top. This may advantageously permit a manufacturer to focus attention on products with which a lot of business is already conducted, which may be useful for purposes of allocating resources or making other decisions.

Additionally or alternatively, information about top vendor representatives 760 may be presented to the manufacturer. For example, top vendor representatives that have the most or highest performance may be displayed to the manufacturer. The top vendor representatives may be calculated based on the number of procedures that are supported, the number of products that have been covered, the length of time that the vendor representative has provided support, the clinical outcomes for procedures where the vendor representatives provide support, feedback or ratings for the vendor representatives, or any other factors or combinations thereof. In some instances, one or more of the factors may be weighted and used to determine a list of top vendor representatives. The top vendor representatives may be displayed so that they are ranked, with the vendor representatives that provide the most and/or best support is displayed at the top. This may advantageously permit a manufacturer to focus attention on top vendor representatives, which may be useful for purposes of rewarding top vendor representatives, or making other decisions.

In some instances, information about engagement over time 970 may be displayed. The engagement over time may explore lengths of relationships with health care facilities, departments, contacts, for various products, with various vendor representatives, or any other factors. Such information may be collected and displayed for any of the factors described elsewhere herein.A user may be able to toggle between views of engagement relating to different products, facilities, locations, etc. In some instances, the engagements may be provided as an overlay so that the engagements for multiple factors can be displayed simultaneously.

The schematic of the user interface is provided by way of example only. Any of the data described herein may be displayed in each of their own visually discernible regions. The regions may be provided adjacent to one another, or above or below one another. The regions may be displayed simultaneously on the same user interface. The regions may have a fixed location or variable location. The regions may have any size or shape. The regions may have any placement relative to one another and are not limited to those provided herein.

For any of the user interfaces, engagement information may be provided. The engagement information may show health care facility engagement increases and/or decreases based on vendor representatives, business units, device manufacturers.

Any of the systems and methods provided herein may be utilized during surgery, post-surgery, and/or pre-surgery. Any description or application provided herein during surgery may also apply to post-surgery and/or pre-surgery, and vice versa.

For example, medical resource management systems may be used after surgery. For instance, data such as the amount of time to recovery after surgery may be collected. The data may include the amount of time until a patient is discharged. The data may include the amount of time until a patient is in stable condition. The data may include the amount of time until a patient is fully recovered or able to re-engage in daily activities. Analysis, ratings, and/or recommendations may be provided based on the recovery data.

The data collected and/or analyzed post-surgery may include the products (e.g., tools, instruments, disposables, etc.) that were used. The data may include the brand/vendor that provided the products. Details of tools and how they were used may be analyzed. This may also affect analysis, ratings and/or recommendations.

The data collected and/or analyzed post-surgery may include the identities of individuals who participated in the medical procedure. For instance, the identities of the medical personnel who were locally present may be collected (e.g., identity of surgeon, nurses, assistants, etc.). This may also include the identities of any remote personnel that may support the medical procedure (e.g., vendor representatives, remote consultants, specialists, technical support, etc.). The analysis, ratings and/or recommendations may also depend on analyzed data relating to identities of individuals.

In some embodiments, the steps that were taken during the procedure may be identified and/or analyzed. The systems and methods provided herein may be able to automatically identity and/or analyze the steps taken. Optionally, recommendations may be provided prior to or during the procedure for steps to take for the procedure. The adherence of the medical personnel to the recommended steps may be analyzed. For instance, if the medical personnel deviates from the recommended steps the systems and methods may identify the deviation and analyze the resulting ramifications. The associated timing information for the steps may be recognized and used during analysis as well. For instance, predicted or recommended timing for the recommended steps may be determined. The actual amount of time the medical personnel takes to perform the steps may be measured and compared with predicted amount of time. Outcomes may be analyzed based on this information. The analysis, ratings and/or recommendations may also depend on analyzed data relating to the steps taken and/or the recommended steps.

The systems and methods provided may measure and/or compare turn around time with a success rate for a surgery. This may be performed with various combinations of factors, such as medical personnel, vendor representatives, other remote support, hospitals, products, steps taken during procedure, adherence to recommended steps, etc. This may be used to analyze which combination of factors may yield the most positive or most negative outcomes.

In some embodiments, medical resource management systems may be used before surgery. For instance, recommendations may be made for medical personnel, remote users, products, hospitals, or other factors for a particular procedure type.

In some instances, a patient's anatomy type and/or medical history may be factored in providing recommendations. For instance, demographic information pertaining to the patient may be used as factors in providing recommendations. Examples of demographic information may include age, gender, ethnicity, geographic location, or other information.

In some embodiments, outcomes, such as success rates, for various medical procedures may be provided and/or displayed. The success rates may be analyzed and/or displayed for various factors individually, or in combination. Examples of such factors may include identities of medical personnel, (e.g., surgeons, anesthesiologists, nurses, assistants, etc.), remote support (e.g., vendor representatives, consultants, specialists, technical support, etc.), health care facilities, products (e.g., tools, instruments, disposables, etc.), steps taken, time spent at each step, accuracy of each step, turn-around time for a procedure, or other factors. The success rates may be viewable by users of a medical console. The success rates pertaining to a particular health care facility may be viewable by administrators or employees of the health care facility. The success rates may be viewable by the public, potential patients, current patients, medical personnel, or other individuals. In some instances, the success rates may only be viewable by a subset of the population that may be granted access. The success rates may be viewable only a medical console in some instances. Optionally, the success rates may be viewable through a web portal.

In some instances, based on any of the factors and analysis provided, the availability of different entities may be displayed. For example, on a medical console or web portal, the availability of different individuals (e.g., medical personnel that are at the location of a medical procedure, remote users) may be determined. In some instances, availability of various resources (e.g., health care facilities, locations within a health care facility (such as operating suite), products (such as tools, instruments, disposables, etc.) may be determined. Availability may be used to book a medical procedure. A medical procedure may be scheduled based on available resources, such as individuals, locations, products, as well as outcomes. In some instances, a combination of factors may be determined that provides an optimal or highly improved outcome for a particular procedure type. The availability of the combination of factors may be assessed to determine a good time to schedule a medical procedure that yields an optimal or improved outcome. In some instances, a user (e.g., medical personnel, administrator, patient) may search for when a combination of factors are available, and schedule a procedure accordingly.

In some embodiments, the medical resource intelligence systems of the present disclosure may be configured to receive, process, update, and/or manage inventory information and/or tool usage information. As used herein, inventory information may comprise information on what types of medical tools, instruments, devices, or resources were previously available, are currently available, or will be available at some point in time. Inventory information may further comprise information on the quantities and availability of such tools, instruments, devices, or resources at different points in time, as well as information on when such tools, instruments, devices, or resources are expected to be used, depleted from stock, or received in a new order or shipment of orders. In some cases, inventory information may comprise information on a historical or projected usage of various tools, instruments, devices, or resources within a certain time frame, or with respect to a particular type of medical procedure, or with respect to a particular doctor, physician, surgeon, or other medical worker. As used herein, tool usage information may comprise information on what types of tools, instruments, devices, or resources have been used, are currently being used, or will be used in the future. In some cases, tool usage information may comprise information on how many tools have been used, are currently in use, or are expected to be used within a certain time frame. In some cases, tool usage information may comprise information on how long the tools have been used or will be used. In some cases, tool usage information may comprise information on what types of tasks or procedures have been completed or will be completed using the tools at some point in time. Tool usage information may correspond to usage of tools that were previously available in inventory, are currently available in inventory, or are expected to be available in inventory at some point in time in the future.

In some cases, the medical resource intelligence systems of the present disclosure may be configured to update or track inventory information based on the tool usage information. For example, the medical resource intelligence system may be configured to update or track inventory information based on a doctor's or surgeon's usage of one or more tools during a medical procedure, based on the preparation of the one or more tools for an upcoming medical procedure, or based on an expected use of one or more tools by a particular doctor or surgeon (e.g., based on a tool preference of the doctor or surgeon). The medical resource intelligence system may be configured to track a usage of one or more tools provided in an operating room (e.g., in a tool tray or a tool cabinet), detect what tools or in the tool tray or tool cabinet have been used or are being used (e.g., based on an optical or image-based detection of the usage of such tools), and update inventory information based on the detected use of the one or more tools. In some cases, tool usage may be detected based on a reading or a scan of one or more identifying features associated with or provided on the tool. The one or more identifying features may comprise, for example, a barcode, a quick response (QR) code, or any other visual pattern or textual data (e.g., alphanumeric sequence). In some cases, tool usage may be detected based on one or more images or videos captured using a camera or imaging sensor located in the operation room. The one or more images or videos may show a usage or a preparation of the tools by a doctor, a surgeon, or other medical worker or assistant before, during, and/or after one or more steps of a surgical procedure. In other cases, tool usage may be detected using a radio-frequency identification (RFID) tag associated with the one or more tools.

In some cases, the medical resource intelligence systems of the present disclosure may be configured to update tool usage information based on a doctor's or surgeon's usage of one or more tools during a medical procedure, or based on the preparation of the one or more tools for an upcoming medical procedure. The medical resource intelligence system may be configured to track a usage of one or more tools provided in an operating room (e.g., in a tool tray or a tool cabinet), and to determine what tools or in the tool tray or tool cabinet have been used or are being used based on an optical or image-based detection of the usage of such tools. In some cases, the optical or image-based detection may comprise identifying the tool based on one or more images or videos captured using a camera or imaging sensor located in the operation room. In some cases, the optical or image-based detection may comprise identifying the tool based on an optical reading or scan of one or more identifying features associated with or provided on the tool. The one or more identifying features may comprise, for example, a barcode, a quick response (QR) code, or any other visual pattern or textual data (e.g., alphanumeric sequence). In some cases, the medical resource intelligence system may be configured to track a usage of one or more tools provided in an operating room (e.g., in a tool tray or a tool cabinet), and to determine what tools in the tool tray or tool cabinet have been used or are being used, based on a radio-frequency identification (RFID) tag associated with the one or more tools.

In some cases, inventory information and/or tool usage information can be updated based on an interaction between a surgeon or medical worker and one or more tools provided in a tool tray or a tool cabinet. The interaction may comprise the surgeon or medical worker lifting a tool from the tool tray, placing the tool back down on the tool tray, repositioning or reorienting the tool relative to the tool tray, adding one or more tools to the tool tray, removing one or more tools from the tool tray, or replacing one or more tools on the tool tray. The inventory information and/or tool usage information can also be updated based on a number of times a tool has been lifted from the tool tray, or a length of time during which the tool is not in contact with the tray (e.g., when the tool is in use by a doctor, a surgeon, a medical worker, or a medical assistant).

In some cases, tool preferences of the surgeon or the healthcare facility for a particular type of procedure may be used to update inventory information or tool usage information. For example, if the surgeon or healthcare facility has a preference for a certain set of tools to be used during one or more steps of a surgical procedure, such preference may be used to update tool usage information or expected tool usage information for one or more upcoming surgical procedures, or for one or more upcoming steps for a surgical procedure. Further, such preference may be used to update inventory information. For example, if a surgeon having a particular tool preference has a procedure scheduled for a certain date, the medical resource intelligence system can update the inventory information based on that surgeon's particular tool preferences. In some cases, the medical resource intelligence system can update the inventory information based on an expected or predicted tool usage. Such expected or predicted tool usage may be determined in part based on the tool preferences of a particular surgeon or a particular healthcare facility in which a medical procedure is to be performed.

In some cases, the tool preferences for a particular surgeon may be determined based on a preference card of the surgeon. In other cases, the tool preferences for a particular surgeon may be determined based on one or more inputs, responses, or instructions provided by the surgeon. In some instances, the tool preferences for a particular surgeon may be determined based on a historical trend or usage of one or more tools by the surgeon for a particular type of surgery.

In some cases, inventory information and tool usage information may be used to determine which tools are in short supply, how many of such tools are in stock, and how many medical procedures can be supported or completed using those tools still available. The medical resource intelligence system may be configured to use the inventory information and/or tool usage information to place or queue an order for one or more additional tools or replacement tools. The medical resource intelligence system may be further configured to use the inventory information and/or tool usage information to provide one or more messages or alerts to a surgeon or a healthcare facility indicating the available stock for one or more tools, and which of the one or more tools are in short supply. In other cases, inventory information and tool usage information may be used to determine which tools are well stocked, how many of such tools are in stock, and how many medical procedures can be supported or completed using those tools currently available. In some cases, the medical resource intelligence system may be configured to use the inventory information and/or tool usage information to order, preorder, or reorder one or more tools based on an expected need for the one or more tools in an upcoming surgical procedure.

Computer Control Systems

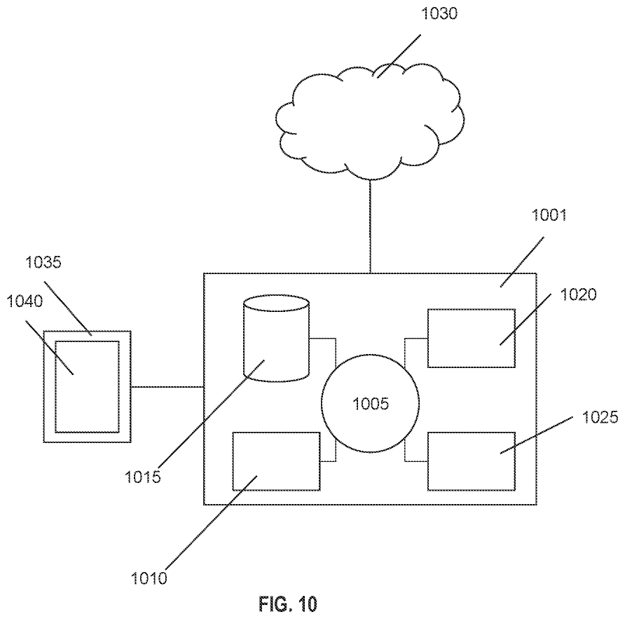

The present disclosure provides computer control systems that are programmed to implement methods of the disclosure. FIG. 10 shows a computer system 1001 that is programmed or otherwise configured to facilitate communications between vendor representatives and medical personnel that may need a vendor representative's support. The computer system may facilitate communications between a rep communication device and a local communication device. The computer system may automatically interface with one or more inventory systems or resource management systems of one or more health care facilities. The computer system may access information about one or more vendors, such as one or more vendor representatives. The computer system may automatically determine one or more vendor representatives that may be suitable for providing support for a medical procedure utilizing a medical product. The computer system may provide medical resource intelligence, such as information about past medical product usage and forecasting future usage. The computer system may provide vendor representative productivity measures. The computer system may advantageously provide engagement information. The computer system can be an electronic device of a user or a computer system that is remotely located with respect to the electronic device. The electronic device can be a mobile electronic device.

The computer system 1001 may include a central processing unit (CPU, also “processor” and “computer processor” herein) 1005, which can be a single core or multi core processor, or a plurality of processors for parallel processing. The computer system also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet.

The network 1030 in some cases is a telecommunication and/or data network. The network can include one or more computer servers, which can enable distributed computing, such as cloud computing. For example, one or more computer servers may enable cloud computing over the network (“the cloud”) to perform various aspects of analysis, calculation, and generation of the present disclosure, such as, for example, capturing a configuration of one or more experimental environments; storing in a registry the experimental environments at each of one or more time points; performing one or more experimental executions which leverage experimental environments; providing outputs of experimental executions which leverage the environments; generating a plurality of linkages between the experimental environments and the experimental executions; and generating one or more execution states corresponding to the experimental environments at one or more time points. Such cloud computing may be provided by cloud computing platforms such as, for example, Amazon Web Services (AWS), Microsoft Azure, Google Cloud Platform, and IBM cloud. The network, in some cases with the aid of the computer system 1001, can implement a peer-to-peer network, which may enable devices coupled to the computer system to behave as a client or a server.

The CPU 1005 can execute a sequence of machine-readable instructions, which can be embodied in a program or software. The instructions may be stored in a memory location, such as the memory 1010. The instructions can be directed to the CPU, which can subsequently program or otherwise configure the CPU to implement methods of the present disclosure. Examples of operations performed by the CPU can include fetch, decode, execute, and writeback.

The CPU 1005 can be part of a circuit, such as an integrated circuit. One or more other components of the system can be included in the circuit. In some cases, the circuit is an application specific integrated circuit (ASIC).

The storage unit 1015 can store files, such as drivers, libraries and saved programs. The storage unit can store user data, e.g., user preferences and user programs. The computer system 1001 in some cases can include one or more additional data storage units that are external to the computer system, such as located on a remote server that is in communication with the computer system through an intranet or the Internet.

The computer system 1001 can communicate with one or more remote computer systems through the network 1030. For instance, the computer system can communicate with a remote computer system of a user (e.g., a user of an experimental environment). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system via the network.

Methods as described herein can be implemented by way of machine (e.g., computer processor) executable code stored on an electronic storage location of the computer system 1001, such as, for example, on the memory 1010 or electronic storage unit 1015. The machine executable or machine readable code can be provided in the form of software. During use, the code can be executed by the processor 1005. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can include or be in communication with an electronic display 1035 that comprises a user interface (UI) 1040 for providing, for example, selection of an environment, a component of an environment, or a time point of an environment. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1005. The algorithm can, for example, capture a configuration of one or more experimental environments; store in a registry the experimental environments at each of one or more time points; perform one or more experimental executions which leverage experimental environments; provide outputs of experimental executions which leverage the environments; generate a plurality of linkages between the experimental environments and the experimental executions; and generate one or more execution states corresponding to the experimental environments at one or more time points.

It should be understood from the foregoing that, while particular implementations have been illustrated and described, various modifications can be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents.

What is claimed is:

1. A method of forecasting usage of one or more medical resources, said method comprising:
(a) collecting, with aid of one or more video or audio systems, images or audio transcripts of one or more medical resources at a health care location;
(b) recognizing, with aid of one or more processors, the one or more medical resources based on the images or audio transcripts collected by the video or audio systems;
(c) assessing, with aid of one or more processors, usage of the one or more medical resources based on the images or audio transcripts collected by the video or audio systems; and
(d) forecasting, with aid of one or more processors, usage of the one or more medical resources based on usage data of the one or more medical resources from multiple health care locations.

2. The method of claim 1, wherein forecasting the usage of the one or more medical resources comprises incorporating one or more health care facility or department rules for the health care location.

3. The method of claim 1, wherein forecasting the usage of the one or more medical resources comprises incorporating usage data collected with aid of one or more medical consoles capable of communicating with a remote device.

4. The method of claim 3, wherein forecasting usage of the one or more medical resources comprises real-time forecasting during a medical procedure at the health care location.

5. The method of claim 1, wherein forecasting the usage of the one or more medical resources comprises incorporating scheduling data from a scheduling system.

6. The method of claim 1, wherein forecasting the usage of the one or more medical resources comprises forecasting a higher usage of a medical product that has a lower cost than a practitioner's preference while providing equivalent or improved outcomes.

7. The method of claim 1, wherein forecasting the usage of the one or more medical resources comprises predicting a future usage of a particular category of product.

8. The method of claim 1, wherein forecasting the usage of the one or more medical resources comprises predicting a future usage of a specific model of product.

9. The method of claim 1, further comprising displaying a forecasted usage of the one or more medical resources to a member of a health care facility.

10. The method of claim 1, further comprising displaying a forecasted usage of the one or more medical resources to a member of a vendor that provides the one or more medical resources.

11. A method of assessing productivity of a remote user providing support during a medical procedure, said method comprising:
(a) collecting, with aid of one or more video or audio systems, information pertaining to support provided by the remote user; and
(b) assessing, with aid of one or more processors, productivity of the remote user based on (i) the information provided by the one or more video and audio systems and (ii) prior support provided by the remote user.

12. The method of claim 11, wherein the one or more video or audio systems comprise a medical console capable of communicating with a remote device of the remote user.

13. The method of claim 11, wherein assessing the productivity of the remote user comprises analyzing data corresponding to an amount of time the remote user spends supporting the medical procedure.

14. The method of claim 13, wherein the amount of time the remote user spends supporting the medical procedure is tracked by detecting a time at which communications are initiated between a medical console and a remote device of the remote user, and a time at which communications are completed between the medical console and the remote device of the remote user.

15. The method of claim 13, wherein the amount of time the remote user spends supporting the medical procedure is tracked by detecting an amount of time during which the remote user is actively interacting with a remote device of the remote user.

16. The method of claim 11, wherein assessing the productivity of the remote user depends on a product or product type supported by the remote user.

17. The method of claim 16, wherein assessing the productivity of the remote user depends on an outcome of the medical procedure utilizing the product or product type supported by the remote user.

18. The method of claim 11, wherein assessing the productivity of the remote user depends on product usage over time for a product supported by the remote user.

19. The method of claim 11, further comprising displaying a quantitative assessment of the remote user's productivity.

20. A system for assessing productivity of a remote user providing support during a medical procedure, said system comprising:
(a) one or more video or audio systems configured to collect information pertaining to support provided by the remote user; and
(b) one or more processors configured to assess, with aid of a machine learning system, productivity of the remote user based on (i) the information provided by the one or more video and audio systems and (ii) prior support provided by the remote user.
